(19) United States
(12) Patent Application Publication
(10) Pub. No.: US 2011/0020329 A1
(43) Pub. Date: Jan. 27, 2011

(54) CONJUGATES OF ANTI-RG-1 ANTIBODIES

(75) Inventors: David J. King, Solana Beach, CA (US); Jonathan A. Terrett, Sunnyvale, CA (US); Sanjeev Gangwar, Foster City, CA (US); Josephine M. Cardarelli, San Carlos, CA (US); Chetana Rao-Naik, Walnut Creek, CA (US); Chin Pan, Los Altos, CA (US)

Correspondence Address: MEDAREX, INC., 521 COTTONWOOD DRIVE, MILPITAS, CA, 95035, US

(73) Assignee: Bristol-Myers Squibb Company

(21) Appl. No.: 12/745,337

(22) PCT Filed: Nov. 26, 2008
(86) PCT No.: PCT/US08/84899

§ 371 Date: May 28, 2010

Related U.S. Application Data
(60) Provisional application No. 60/991,690, filed on Nov. 30, 2007.

(51) Int. Cl. A61K 39/395; C07K 16/18; C12N 5/09; A61P 35/00

(52) U.S. Cl. 424/133.1; 530/387.3; 435/325

(57) ABSTRACT

Anti-RG-1 antibodies, antibody fragments, or antibody mimetics conjugated to partner molecules, such as drugs, radioisotopes, and cytotoxins, wherein the partner molecule exerts its effect regardless of whether the RG-1 bound conjugate is internalized within a targeted cell, are useful for treating cancers.

### TECHNICAL FIELD

The present invention provides anti-RG-1 antibodies conjugated to partner molecules wherein the partner molecule exerts its effect regardless of whether the bound RG-1 is internalized within a targeted cell, for the treatment of diseases such as cancer.

### BACKGROUND ART

Antibody-partner molecules conjugated to cytotoxic compounds have been developed for the treatment of diseases, including cancer. Conventionally, this technology has been restricted to antigen targets where the antibody/antigen complex is internalized into the target cell and the partner molecule is then released and/or activated intracellularly. Such conjugates are also known generically as antibody-drug conjugates, or ADCs.

An example of a disease that may be treated using such an “internalization-based” system is prostate cancer. Prostate cancer is common disease in men, affecting about one third of men over the age of 45. There is evidence for both genetic and environmental causes, with the majority of cases probably being the result of a combination of both factors.

Prostate cancer is usually diagnosed by physical examination and by serum levels of prostate specific antigen (PSA). Radical prostatectomy is the treatment of choice for localized disease. Advanced metastatic disease is treated currently by androgen ablation therapy or treatment with GnRH (gonadotrophin releasing hormone) and by anti-androgen therapy. However, advanced disease almost invariably becomes hormone resistant and there is no cure for progressive disease. Moreover, there are serious side effects associated with both radical prostatectomy and androgen ablation therapy. While internalization-based systems may hold promise as an alternative to such therapeutic approaches, there remains a considerable need for new therapeutic approaches for both early and late stage prostate cancer.

The polypeptide RG-1 is a homolog of the Mindin/F-spondin family of extracellular matrix proteins. (U.S. Pat. No. 5,871,969). It is highly expressed in prostate tissue (WO 98/45442) and therefore should be a useful target for the diagnosis and therapy of prostate cancer and other cancers where it is expressed, such as bladder cancer. Harkins et al., U.S. Pat. No. 7,335,748 discloses human anti-RG-1 antibodies and their use in the detection and treatment of cancer. We have discovered that, unexpectedly, conjugates of RG-1 antibodies and a partner molecule are effective for treating disorders associated with aberrant RG-1 expression, despite the fact that RG-1 is not internalized upon antibody binding.

### DISCLOSURE OF THE INVENTION

The present disclosure provides isolated anti-RG-1 antibody-partner molecule conjugates that specifically bind to RG-1 with high affinity. This disclosure also provides methods for treating cancers, such as prostate and bladder cancers, using such conjugates.

In one embodiment, an antibody-partner molecule conjugate comprises an antibody or an antigen-binding portion thereof conjugated to a partner molecule, wherein the antibody or antigen-binding portion thereof binds human RG-1 and the conjugate exhibits at least one (and preferably both) of the following properties:
(a) binds to human RG-1 with a K_{D} of 1×10⁻⁸ M or less; or
(b) inhibits growth of RG-1-expressing cells in vivo.

Preferably, the antibody portion of the conjugate is a human antibody, more preferably a human monoclonal antibody. Also preferably, the antibody or antigen binding portion thereof cross-competes for binding to an epitope on human RG-1 that is recognized by a reference antibody that comprises:
(a) a heavy chain variable region (V_{H}) comprising the amino acid sequence of SEQ ID NO: 13 and a light chain variable region (V_{L}) comprising the amino acid sequence of SEQ ID NO: 15; or
(b) a V_{H} comprising the amino acid sequence of SEQ ID NO: 14 and a V_{L} comprising the amino acid sequence of SEQ ID NO: 16.

In a preferred embodiment, the reference antibody comprises a V_{H} comprising the amino acid sequence of SEQ ID NO: 13 and a V_{L} comprising the amino acid sequence of SEQ ID NO: 15. In another preferred embodiment, the reference antibody comprises a V_{H} comprising the amino acid sequence of SEQ ID NO: 14 and a V_{L} comprising the amino acid sequence of SEQ ID NO: 16.

A particularly preferred antibody, or antigen-binding portion thereof, of a conjugate of the instant invention comprises:
(a) a V_{H} CDR1 comprising SEQ ID NO: 1;
(b) a V_{H} CDR2 comprising SEQ ID NO: 3;
(c) a V_{H} CDR3 comprising SEQ ID NO: 5;
(d) a V_{L} CDR1 comprising SEQ ID NO: 7;
(e) a V_{L} CDR2 comprising SEQ ID NO: 9; and
(f) a V_{L} CDR3 comprising SEQ ID NO: 11.

Another particularly preferred antibody, or antigen-binding portion thereof, of a conjugate of the instant invention comprises:
(a) a V_{H} CDR1 comprising SEQ ID NO: 2;
(b) a V_{H} CDR2 comprising SEQ ID NO: 4;
(c) a V_{H} CDR3 comprising SEQ ID NO: 6;
(d) a V_{L} CDR1 comprising SEQ ID NO: 8;
(e) a V_{L} CDR2 comprising SEQ ID NO: 10; and
(f) a V_{L} CDR3 comprising SEQ ID NO: 12.

In another aspect, the monoclonal antibody, or antigen binding portion thereof, of a conjugate of this invention comprises:
(a) a V_{H} comprising an amino acid sequence selected from the group consisting of SEQ ID NOs: 13 or 14; and
(b) a V_{I}, comprising an amino acid sequence selected from the group consisting of SEQ ID NOs: 15 or 16;
wherein the antibody specifically binds human RG-1.

A preferred combination of the foregoing comprises:
(a) a V_{H} comprising the amino acid sequence of SEQ ID NO: 13; and
(b) a V_{L} comprising the amino acid sequence of SEQ ID NO: 15.

Another preferred combination of the foregoing comprises:
(a) a V_{H} comprising the amino acid sequence of SEQ ID NO: 14; and
(b) a V_{L} comprising the amino acid sequence of SEQ ID NO: 16.

The antibodies can be full-length ones such as of the IgG1 or IgG4 isotype, or antibody fragments, such as Fab, Fab′ or Fab′2 fragments, or single chain antibodies.

In another aspect, the invention pertains to a method of inhibiting growth of a RG-1-expressing tumor cell, comprising contacting the cell with an antibody-partner molecule conjugate of the disclosure such that growth of the RG-1-tumor cell is inhibited.

In another aspect, the invention pertains to a method of treating cancer in a subject in need of such treatment, comprising administering to the subject an effective amount of an antibody-partner molecule conjugate of the disclosure such that the cancer is treated in the subject. Preferably, the cancer is one characterized by RG-1 expressing cells.

### BRIEF DESCRIPTION OF THE DRAWING(S)

FIG. 1A shows the nucleotide (SEQ ID NO: 17) and amino acid (SEQ ID NO: 13) sequences of the V_{H} of the 19G9 human monoclonal antibody. The CDR1 (SEQ ID NO: 1), CDR2 (SEQ ID NO: 3) and CDR3 (SEQ ID NO: 5) regions are delineated.

FIG. 1B shows the nucleotide (SEQ ID NO: 19) and amino acid (SEQ ID NO: 15) sequences of the V_{L} of the 19G9 human monoclonal antibody. The CDR1 (SEQ ID NO: 7), CDR2 (SEQ ID NO: 9) and CDR3 (SEQ ID NO: 11) regions are delineated.

FIG. 2A shows the nucleotide (SEQ ID NO: 18) and amino acid (SEQ ID NO: 14) sequences of the V_{H} of the 34E1 human monoclonal antibody. The CDR1 (SEQ ID NO: 2), CDR2 (SEQ ID NO: 4) and CDR3 (SEQ ID NO: 6) regions are delineated.

FIG. 2B shows the nucleotide (SEQ ID NO: 20) and amino acid (SEQ ID NO: 16) sequences of the V_{L} of the 34E1 human monoclonal antibody. The CDR1 (SEQ ID NO: 8), CDR2 (SEQ ID NO: 10) and CDR3 (SEQ ID NO: 12) regions are delineated.

FIGS. 3A and 3B show the EC_{S}, values of in vitro tumor-activated activity of certain antibody-partner molecule conjugates on LNCaP and 786-O cells, respectively.

FIGS. 4A-4D and 5A-5D show the results of an in vivo LNCaP/prostate stroma cell xenograft mouse model, presenting median tumor volume (FIGS. 4A-4D) and median body weight change data (FIGS. 5A-5D).

FIGS. 6A-6D and 7A-7D show the results of an in vivo LNCaP xenograft mouse model, presenting median tumor volume (FIGS. 6A-6D) and median body weight change data (FIGS. 7A-7D).

### MODE FOR CARRYING OUT THE INVENTION

The present invention relates to antibodies, antibody fragments, and antibody mimetics that bind specifically and with high affinity to RG-1 and are conjugated to partner molecules that do not require internalization of the conjugate to exert their effectiveness.

Non-internalized antigens such as RG-1 are retained at a tumor site and are not rapidly internalized upon binding of the antibody. It has been reported that conjugate efficacy is dependent upon antibody-antigen interaction at the cell surface triggering the internalization, trafficking and subsequent release of the active cytotoxic payload (Sutherland et al., J. Biol. Chem. 281, 10540-10547 (2006)). However, in this invention we demonstrate that retention of antibody drug conjugates at the tumor site is sufficient to result in cytotoxic effects to the tumor, even though the antigen being targeted is not internalized, or may not be present on the tumor cells themselves, but alternatively on surrounding extracellular matrix, stromal cells, tumor vasculature cells, or invading inflammatory cells, such as tumor associated macrophages. Alternatively, retention can occur when the antigen is shed from the tumor cells but is retained at the tumor site by its association with tumor cells, the extracellular matrix, stromal cells, invading inflammatory cells or tumor vasculature cells.

Non-internalized antigens, such as RG-1, are retained a tumor site and are not internalized upon the binding of an antibody. Retention of such antigens at the tumor site can be mediated by attachment of the target antigen to the external plasma membrane of the tumor cell, surrounding stromal cells, or tumor vasculature cells. Alternatively, retention can occur when the antigen is shed from tumor cells but is retained at the tumor site by its association with the extracellular matrix or tumor, stromal, or tumor vasculature cells.

In the case of an antibody-partner molecule conjugate, it will be held at the disease site by antigen binding, enabling tumor-biased release of the partner molecule. Upon the partner molecule's release (e.g., by cleavage of a linker group as described hereinbelow), it can then pass freely into the neighboring cells, become activated, and exert its effects. Cleavage of the linker group can be take advantage of the lower extracellular pH (pHe) of tumors, which is commonly around 6.8, or about 0.5 units lower than that of normal tissue, in the case of pH sensitive linkers such as hydrazones. Or, the linker can be cleaved by proteases in the extracellular matrix of a tumor or on the surface of cells in the tumor, such as CD10, cathepsins, matrix metalloproteases, and serine proteases.

In order that the present invention may be more readily understood, certain terms are first defined. Additional definitions are set forth throughout the detailed description.

The term “RG-1” includes variants, isoforms, and species homologs of human RG-1. Accordingly, human antibodies of this disclosure may, in certain cases, cross-react with RG-1 from species other than human. In certain embodiments, the antibodies, antibody fragments, or antibody mimetics may be completely specific for one or more human RG-1 and may not exhibit species or other types of non-human cross-reactivity.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

A “signal transduction pathway” refers to the biochemical relationship between various signal transduction molecules that play a role in the transmission of a signal from one portion of a cell to another portion of a cell. The phrase “cell surface receptor” includes molecules and complexes of molecules capable of receiving a signal and the transmission of such a signal across the plasma membrane of a cell, an example being the RG-1 receptor.

The term “antibody” refers to whole antibodies and any antigen binding fragment (“antigen-binding portion”) or single chains thereof. A “full length antibody” refers to a protein comprising at least two heavy (H) chains and two light (L) chains inter-connected by disulfide bonds. Each heavy chain comprises a heavy chain variable region (abbreviated as V_{H}) and a heavy chain constant region. The heavy chain constant region comprises three domains, C_{H}1, C_{H}2 and C_{H}3. Each light chain comprises a light chain variable region (abbreviated as V_{L}) and a light chain constant region. The light chain constant region comprises one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The terms “antibody fragment” and “antigen-binding portion” of an antibody (or simply “antibody portion”) refer to one or more fragments of an antibody that retain the ability to specifically bind to an antigen (e.g., RG-1), it having been shown that the antigen-binding function can be performed by fragments of a full-length antibody. Examples of such binding fragments include (i) a Fab fragment, a monovalent fragment consisting of the V_{L}, V_{H}, C_{L and C}_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a V_{H} containing a single variable domain and two constant domains. Although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody.

An “isolated antibody” refers to an antibody that is substantially free of other antibodies having different antigenic specificities (e.g., an isolated antibody that specifically binds RG-1 is substantially free of antibodies that specifically bind antigens other than RG-1). An isolated antibody that specifically binds RG-1 may, however, have cross-reactivity to other antigens, such as RG-1 molecules from other species. Moreover, an isolated antibody may be substantially free of other cellular material and/or chemicals.

The terms “monoclonal antibody” or “monoclonal antibody composition” refer to a preparation of antibody molecules of single molecular composition. A monoclonal antibody composition displays a single binding specificity and affinity for a particular epitope.

The term “human antibody” includes antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity having variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and an antibody specific for an antigen” are used interchangeably herein with the term “an antibody that binds specifically to an antigen.”

The term “humanized antibody” refers to antibodies in which CDR sequences derived from the germline of another mammalian species, such as a mouse, have been grafted onto human framework sequences. Additional framework region modifications may be made within the human framework sequences.

The term “chimeric antibody” refers to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” refers to molecules capable of mimicking an antibody's ability to bind an antigen, but which are not limited to native antibody structures. Examples of such antibody mimetics include, but are not limited to, Affibodies, DARPins, Anticalins, Avimers, and Versabodies.

The term “partner molecule” refers to the entity that is conjugated to an antibody in an antibody-partner molecule conjugate. Examples of partner molecules include drugs, cytotoxins, marker molecules (including, but not limited to peptide and small molecule markers such as fluorochrome markers, as well as single atom markers such as radioisotopes), proteins and therapeutic agents.

As used herein, an antibody that “specifically binds to human RG-1” refers to an antibody that binds to human RG-1 with a K_{D} of 1×10⁻⁷ M or less, more preferably 5×10⁻⁸ M or less, more preferably 3×10⁻⁸ M or less, more preferably 1×10⁻⁸ M or less, even more preferably 5×10⁻⁹ M or less.

The term “does not substantially bind” to a protein or cell means that the antibody does not bind with a high affinity to a protein or a cell, i.e. binds to the protein or cell with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

The term “high affinity” for an IgG antibody refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (e.g., C₁-C₁₀ means one to ten carbons). Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, n-pentyl, n-hexyl, n-heptyl, n-octyl, and the like, and homologs and isomers thereof. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, also includes those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, and S, and wherein the nitrogen, carbon and sulfur atoms may optionally be oxidized and the nitrogen heteroatom may optionally be quaternized. The heteroatom(s) O, N, S, and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(═O)—CH₃, —CH₂—CH₂—S(═O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula —CO₂R % represents both —CO₂R′— and —R′CO₂—.

The term “lower” in combination with the terms “alkyl,” “alkylene,” “heteroalkyl,” or the like refers to a moiety having from 1 to 6 carbon atoms.

The terms “alkoxy,” “alkylamino,” “alkylsulfonyl,” and “alkylthio” (or thioalkoxy) are used in their conventional sense, and refer to those alkyl groups attached to the remainder of the molecule via an oxygen atom, an amino group, an SO₂ group or a sulfur atom, respectively. The term “arylsulfonyl” refers to an aryl group attached to the remainder of the molecule via an SO₂ group, and the term “suithydryl” refers to an SH group.

The term “acyl substituent” refers to groups attached to, and fulfilling the valence of a carbonyl carbon that is either directly or indirectly attached to the polycyclic nucleus of the compounds of the present invention. The substituent portion of “acyl substituent” can be selected from the groups set forth above.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen,” by themselves or as part of another substituent, mean a fluorine, chlorine, bromine, or iodine atom. Additionally, terms such as “haloalkyl,” include monohaloalkyl and polyhaloalkyl. Thus, “halo(C₁-C₄)alkyl” includes, but is not limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents,” respectively, and they can be one or more of a variety of groups selected from, but not limited to: —OR′, ═O, ═NR′, —NR′R″, —SR′, -halogen, —SiR′R″R′″, —OC(═O)R′, —C(═O)R′, —CO₂R′, —CONR′R″, —OC(═O)NR′R″, —NR″C(═O)R′, —NR′—C(═O)NR″R′″, —NR″CO₂R′, —NR—C(NR′R″R′″)═NR′″, —NR—C(NR′R″)═NR′″, —S(═O)R′, —S(═O)₂R′, —S(═O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to faun a 5, 6, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(═O)CH₃, —C(═O)CF₃, —C(═O)CH₂OCH₃, and the like).

Similarly to the substituents described for the alkyl radical, the aryl substituents and heteroaryl substituents are generally referred to as “aryl substituents” and “heteroaryl substituents,” respectively, and are varied and selected from, e.g.: halogen, —OR′, ═O, ═NR′, ═N—OR′, —NR′R″, —SR′, -halogen, —SiR′R″R′″, —OC(═O)R′, —C(═O)R′, —CO₂R′, —CONR′R″, —OC(═O)NR′R″, —NR″C(═O)R′, —NR′—C(═O)NR″R′″, —NR″CO₂R′, —NR—C(NR′R″)═NR′″, —S(═O)R′, —S(═O)₂R′, —S(═O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R, R′, R″, R′″, or R″″ group, each such group is variable independent of the other(s).

Two substituents on adjacent atoms of an aryl or heteroaryl ring may optionally be replaced with a substituent of the formula -T-C(═O)—(CRR′)_{q}—U—, wherein T and U are independently —NR—, —O—, —CRR′— or a single bond, and q is an integer from 0 to 3. Or, two of such substituents may optionally be replaced with a substituent of the formula -A-(CH₂)ᵣ—B—, wherein A and B are independently —CRR′—, —O—, —NR—, —S—, —S(═O)—, —S(═O)₂—, —S(═O)₂NR′— or a single bond, and r is an integer from 1 to 4. One of the single bonds of the new ring so formed may optionally be replaced with a double bond. Alternatively, two of such substituents may optionally be replaced with a substituent of the formula —(CRR′)ₙ—X—(CR″R′″)_{d}—, where s and d are independently integers of from 0 to 3, and X is —O—, —NR′—, —S—, —S(═O)—, —S(═O)₂—, or —S(═O)₂NR′—. The substituents R, R′, R″ and R′ are preferably independently selected from hydrogen and substituted or unsubstituted (C₁-C₆) alkyl.

As used herein, the term “diphosphate” includes but is not limited to an ester of phosphoric acid containing two phosphate groups. The term “triphosphate” includes but is not limited to an ester of phosphoric acid containing three phosphate groups.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

Various aspects of the invention are described in further detail in the following subsections.

### Anti-RG-1 Antibodies

The antibodies in the conjugates of the invention are characterized by specific binding to human RG-1. Preferably, the antibody binds to RG-1 with high affinity, for example with a K_{D} of 1×10⁻⁷ M or less. The anti-RG-1 antibodies preferably exhibit one or more of the following characteristics:
(a) binds to human RG-1 with a K_{D} of 1×10⁻⁷ M or less;
(b) binds to CHO cells transfected with RG-1; and
(c) inhibits growth of RG-1-expressing cells in vivo.

In a preferred embodiment, the antibody exhibits at least two of properties (a), (b), and (c). In a more preferred embodiment, the antibody exhibits all three of properties (a), (b), and (c). Preferably, the antibody binds to human RG-1 with a K_{D} of 5×10⁻⁸ M or less, binds to human RG-1 with a K_{D} of 2×10⁻⁸ M or less, binds to human RG-1 with a K_{D} of 5×10⁻⁹ M or less, binds to human RG-1 with a K_{D} of 4×10⁻⁹M or less, binds to human RG-1 with a K_{D} of 3×10⁻⁹ M or less, or binds to human RG-1 with a K_{D} of 2×10⁻⁹ M or less.

The antibody preferably binds to an antigenic epitope in RG-1 that is not present in other proteins. The antibody preferably binds to RG-1 but not to other proteins, or binds to other proteins with a low affinity, such as with a K_{D} of 1×10⁻⁶M or more, more preferably 1×10⁻⁵ M or more, more preferably 1×10⁻⁴ M or more, more preferably 1×10⁻³ M or more, and even more preferably 1×10⁻² M or more.

Standard assays to evaluate the binding ability of the antibodies toward RG-1 are known in the art, including for example, ELISAs, Western blots, RIAs, and flow cytometry analysis. Binding also can be assessed by standard assays, such as by Biacore® system analysis. To assess binding to Raji or Daudi B cell tumor cells, Raji (ATCC Deposit No. CCL-86) or Daudi (ATCC Deposit No. CCL-213) cells can be obtained from the American Type Culture Collection.

### Monoclonal Antibodies 19G9 and 34E1

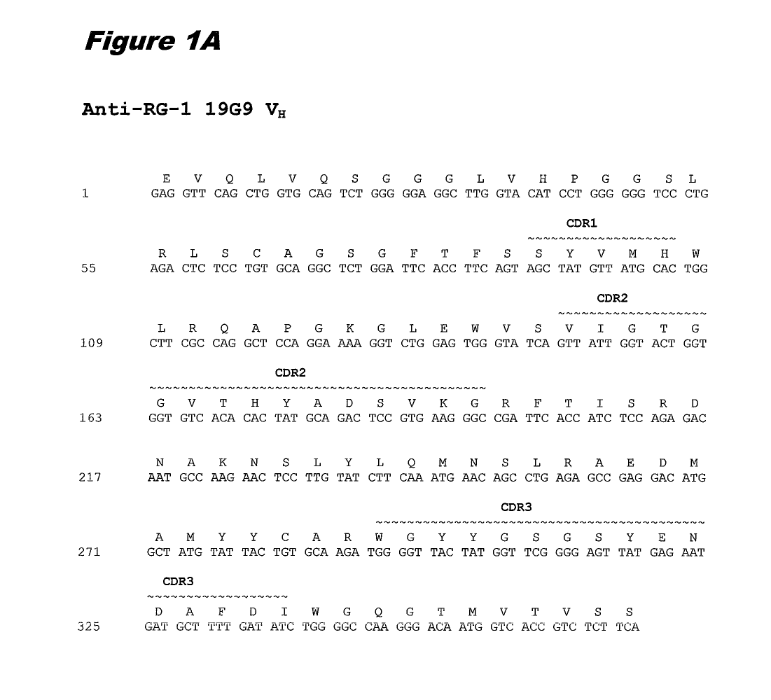

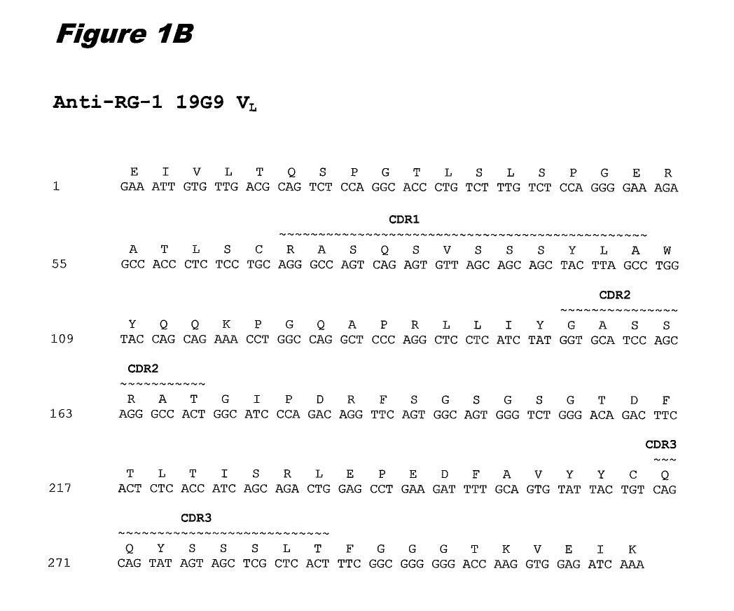

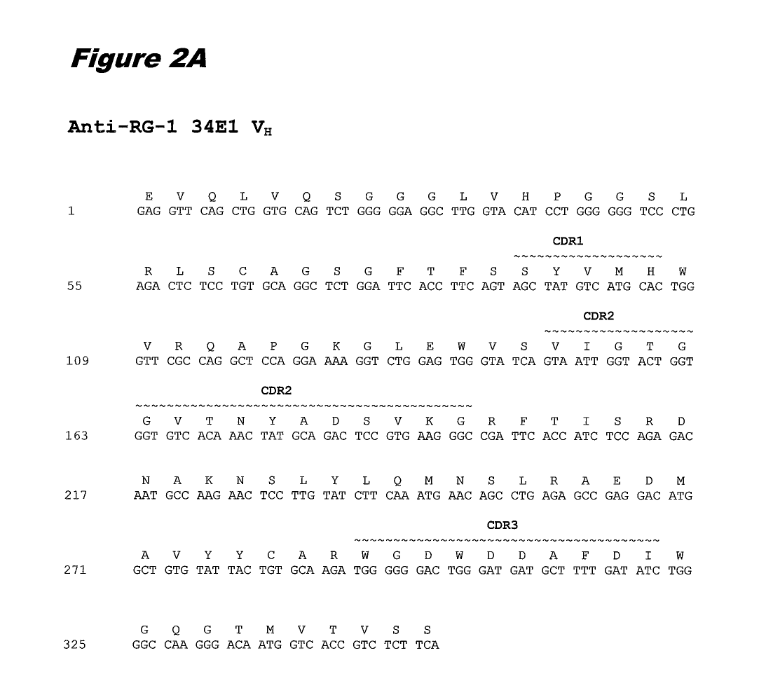

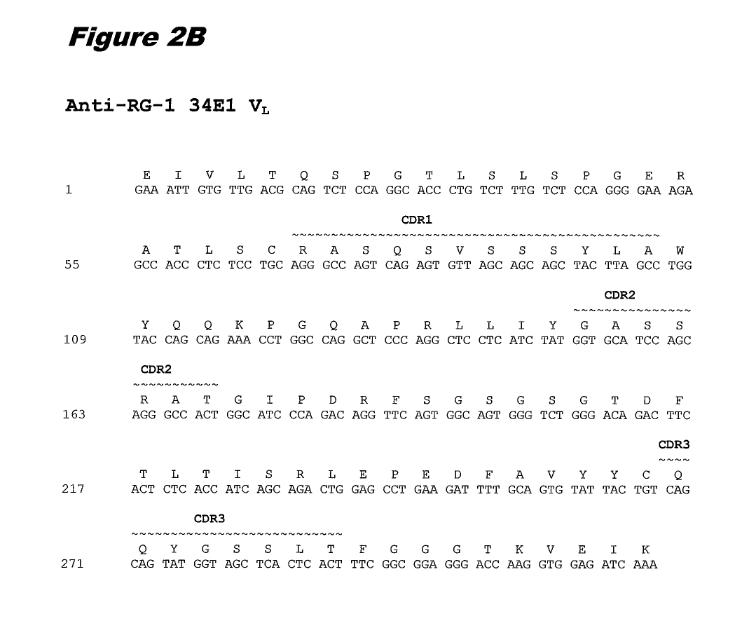

Preferred antibodies for use in conjugates of this disclosure are the human monoclonal antibodies 19G9 and 34E1, which are described in US 2004/0152139 and U.S. Pat. No. 7,335,748 B2, which are hereby incorporated by reference. The V_{H} amino acid sequences of 19G9 and 34E1 are shown in SEQ ID NOs: 13 (FIG. 1A) and 14 (FIG. 2A), respectively. The V_{L} amino acid sequences of 19G9 and 34E1 are shown in SEQ ID NOs: 15 (FIG. 1B) and 16 (FIG. 2B), respectively. The corresponding encoding nucleotide sequences are also shown in the aforementioned figures: SEQ ID NO: 17 (FIG. 1A) for V_{H} of antibody 19G9, SEQ ID NO: 19 (FIG. 1B) for V_{L} of antibody 19G9, SEQ ID NO: 18 (FIG. 2A) for V_{H} of antibody 34E1, and SEQ ID NO: 20 (FIG. 2B) for V_{L} of antibody 34E1.

In another aspect, the antibodies can comprise the heavy chain and light chain CD1s, CDR2s and CDR3s of 19G9 or 34E1, or combinations thereof. The amino acid sequences of the V_{H} CDR1s of 19G9 and 34E1 are shown in SEQ ID NOs: 1-2, respectively. The amino acid sequences of the V_{H} CDR2s of 19G9 and 34E1 are shown in SEQ ID NOs: 3-4, respectively. The amino acid sequences of the V_{H} CDR3s of 19G9 and 34E1 are shown in SEQ ID NOs: 5-6, respectively. The amino acid sequences of the V_{L} CDR1s of 19G9 and 34E1 are shown in SEQ ID NOs: 7-8, respectively. The amino acid sequences of the V_{L} CDR2s of 19G9 and 34E1 are shown in SEQ ID NOs: 9-10. The amino acid sequences of the V_{L} CDR3s of 19G9 and 34E1 are shown in SEQ ID NOs: 11-12, respectively. The CDR regions are delineated using the Kabat system (Kabat, E. A., et al. (1991). Sequences of Proteins of Immunological Interest, Fifth Edition, U.S. Department of Health and Human Services, NIH Publication No. 91-3242; hereinafter “Kabat '3242”).

Because antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and matched, although each antibody must contain a V_{H} CDR1, CDR2, and CDR3 and a V_{L} CDR1, CDR2, and CDR3) to create other anti-RG-1 binding molecules. Preferably, in mixing and matching, a V_{H} sequence from a particular V_{H}/V_{L} pairing is replaced with a structurally similar V_{H} sequence. Likewise, a V_{L} sequence from a particular V_{H}/V_{L} pairing preferably is replaced with a structurally similar V_{L} sequence. RG-1 binding of such “mixed and matched” antibodies can be tested using the binding assays described above. Preferably, when V_{H} CDR sequences are mixed and matched, the CDR1, CDR2 and/or CDR3 sequence from a particular V_{H} sequence is replaced with a structurally similar CDR sequence. Likewise, when V_{L} CDR sequences are mixed and matched, the CDR1, CDR2 and/or CDR3 sequence from a particular V_{L} sequence preferably is replaced with a structurally similar CDR sequence. It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 19G9 and 34E1.

Accordingly, in another aspect, the antibody or antigen-binding portion thereof of a conjugate of this invention comprises:
(a) a heavy chain variable region CDR1 comprising SEQ ID NO: 1 or SEQ ID NO: 2;
(b) a heavy chain variable region CDR2 comprising SEQ ID NO: 3 or SEQ ID NO: 4;
(c) a heavy chain variable region CDR3 comprising SEQ ID NO: 5 or SEQ ID NO: 6;
(d) a light chain variable region CDR1 comprising SEQ ID NO: 7 or SEQ ID NO: 8;
(e) a light chain variable region CDR2 comprising SEQ ID NO: 9 or SEQ ID NO: 10; and
(f) a light chain variable region CDR3 comprising SEQ ID NO: 11 or SEQ ID NO: 12.

It is well known that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116, 2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8:Scientific Review 8 (2001); Igarashi et al., J. Biochem (Tokyo) 117:452-7 (1995); Bourgeois et al., J. Virol 72:807-10 (1998); Levi et al., Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these documents is hereby incorporated by reference in its entirety.

Accordingly, the present invention provides monoclonal antibodies comprising one or more heavy and/or light chain CDR3s from an antibody derived from a human or non-human animal, wherein the monoclonal antibody is capable of specifically binding to human RG-1. In certain aspects, the conjugates of the present disclosure can use monoclonal antibodies comprising one or more heavy and/or light chain CDR3 domain from a non-human antibody, such as a mouse or rat antibody, wherein the monoclonal antibody is capable of specifically binding to RG-1. Or, they can comprise one or more heavy and/or light chain CDR3 domain from a non-human antibody that (a) are capable of competing for binding with; (b) retain the functional characteristics of; (c) bind to the same epitope as; and/or (d) have a similar binding affinity as the corresponding parental non-human antibody.

### Antibodies Having Particular Germline Sequences

In certain embodiments, the antibody portion of a conjugate of this invention comprises a V_{H} from a particular germline heavy chain immunoglobulin gene and/or a V_{L} from a particular germline light chain immunoglobulin gene.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. Such human antibody can be identified by comparing its amino acid sequence with the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody in the antibody portion of a conjugate of this invention comprises heavy and light chain variable regions comprising amino acid sequences that are homologous to the amino acid sequences of the preferred antibodies described herein, and wherein the antibodies retain the desired functional properties of the anti-RG-1 antibodies of the invention.

For example, this disclosure provides an isolated monoclonal antibody, or antigen binding portion thereof, comprising a V_{H} and a V_{L}, wherein:
(a) the V_{H} comprises an amino acid sequence that is at least 80% homologous to an amino acid sequence selected from the group consisting of SEQ ID NOs: 13-14;
(b) the V_{L} comprises an amino acid sequence that is at least 80% homologous to an amino acid sequence selected from the group consisting of SEQ ID NOs: 15-16; and
(c) the antibody specifically binds to human RG-1.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 17-18 or 19-20, followed by testing of the encoded altered antibody for retained function (i.e., the functions set forth above) using the functional assays described herein.

The percent homology between two amino acid sequences is equivalent to the percent identity between the two sequences, which is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### Antibodies Having Conservative Modifications

Antibodies used in conjugates of this invention can comprise a V_{H} comprising CDR1, CDR2 and CDR3 sequences and a V_{L} comprising CDR1, CDR2 and CDR3 sequences, wherein one or more of these CDR sequences comprise specified amino acid sequences based on known anti-RG-1 antibodies, or conservative modifications thereof, and wherein the antibodies retain the desired functional properties of the anti-RG-1 antibodies of this disclosure. It is understood in the art that certain conservative sequence modification can be made which do not remove antigen binding. See, for example, Brummell et al. (1993) Biochem 32:1180-8; de Wildt et al. (1997) Prot. Eng. 10:835-41; Komissarov et al. (1997) J. Biol. Chem. 272:26864-26870; Hall et al. (1992) J. Immunol. 149:1605-12; Kelley and O'Connell (1993) Biochem. 32:6862-35; Adib-Conquy et al. (1998) Int. Immunol. 10:341-6 and Beers et al. (2000) Clin. Can. Res. 6:2835-43. Accordingly, a conjugate of this invention can comprise an antibody, or antigen-binding portion thereof, that specifically binds human RG-1, wherein:
(a) the V_{H} CDR3 sequence comprises the amino acid sequence of SEQ ID NOs: 5-6; and
(b) the V_{L} CDR3 sequence comprises the amino acid sequence of SEQ ID NOs: 11-12;
at least one of V_{H} CDR3 and V_{L} CDR3 having one or more conservative modifications thereto.

Additionally or alternatively, the antibody may possess one or more of the following functional properties described above, such as high affinity binding to human RG-1, the ability to bind CHO cells transfected with RG-1, and/or the ability to inhibit tumor growth of RG-1-expressing tumor cells in vivo when conjugated to a cytotoxin.

In a preferred embodiment, the V_{H} CDR2 sequence comprises an amino acid sequence selected from SEQ ID NOs: 3-4; the V_{L} CDR2 sequence comprises an amino acid sequence selected from SEQ ID NOs: 9-10; the V_{H} CDR1 sequence comprises an amino acid sequence selected from SEQ ID NOs: 1-2; and/or the V_{L} CDR1 sequence comprises an amino acid sequence selected from SEQ ID NOs: 7-8, where one or more of the aforesaid V_{H} CDR2, V_{L} CDR2, V_{H} CDR1, and V_{L} CDR1 can have conservative modifications thereto.

Accordingly, in another aspect, the antibody or antigen-binding portion thereof of a conjugate of this invention comprises:
(a) a heavy chain variable region CDR1 comprising SEQ ID NO: 1 or SEQ ID NO: 2 or conservative modifications of either;
(b) a heavy chain variable region CDR2 comprising SEQ ID NO: 3 or SEQ ID NO: 4 or conservative modifications of either;
(c) a heavy chain variable region CDR3 comprising SEQ ID NO: 5 or SEQ ID NO: 6 or conservative modification of either;
(d) a light chain variable region CDR1 comprising SEQ ID NO: 7 or SEQ ID NO: 8 or conservative modifications of either;
(e) a light chain variable region CDR2 comprising SEQ ID NO: 9 or SEQ ID NO: 10 or conservative modifications of either; and
(f) a light chain variable region CDR3 comprising SEQ ID NO: 11 or SEQ ID NO: 12 or conservative modifications of either.

The term “conservative sequence modifications” refers to amino acid modifications that do not significantly affect or alter the binding characteristics of the antibody containing the amino acid sequence and include amino acid substitutions, additions and deletions. Modifications can be introduced into an antibody of the invention by standard techniques, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (a) through (c) above) using the functional assays described herein. Preferably, the conservative modifications are no more than one or two in number.

Antibodies that Bind to the Same Epitope as Anti-RG-1 Antibodies

In another embodiment, the antibody in a conjugate of this invention binds an epitope on RG-1 recognized by any of the anti-RG-1 monoclonal antibodies of this disclosure (i.e., antibodies that have the ability to cross-compete for binding to human RG-1 with any of the monoclonal antibodies of this disclosure). In preferred embodiments, the reference antibody for cross-competition studies is antibody 19G9 or antibody 34E1.

Cross-competing antibodies can be identified based on their ability to cross-compete with 19G9 or 34E1 in standard PG-1 binding assays. For example, ELISA assays can be used in which a recombinant human RG-1 protein is immobilized on the plate, one of the antibodies is fluorescently labeled and the ability of non-labeled antibodies to compete against the labeled antibody is evaluated. Additionally or alternatively, BIAcore analysis can be used to assess the ability of the antibodies to cross-compete. In a preferred embodiment, the antibody that binds to the same epitope on human RG-1 as is recognized by 19G9 or 34E1 is a human monoclonal antibody. Such human monoclonal antibodies can be prepared and isolated using methods described herein and those known in the art.

### Engineered and Modified Antibodies

An antibody in a conjugate of this invention also can be prepared from an antibody having one or more known V_{H} and/or V_{L} sequences as starting material to engineer a modified antibody that has altered properties compared to the starting antibody. An antibody can be engineered by modifying one or more amino acids within one or both variable regions, for example within one or more CDR regions and/or within one or more framework regions. Additionally or alternatively, an antibody can be engineered by modifying residues within the constant region(s), for example to alter the effector function(s).

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germline antibody gene sequences. For example, germline DNA sequences for human heavy and V_{L} genes can be found in the “VBase” human germline sequence database (available on the Internet at www.mrc-cpe.cam.ac.uk/vbase), as well as in Kabat '3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the con-tents of each of which are expressly incorporated herein by reference. Also, the germline DNA sequences for human heavy and V_{L} genes can be found in the Genbank database. For example, the following heavy chain germline sequences found in the HCo7 HuMAb mouse are available in the accompanying Genbank accession numbers: 1-69 (NG_{—}0010109, NT_{—}024637 and BC070333), 3-33 (NG_{—}0010109 and NT_{—}024637) and 3-7 (NG_{—}0010109 and NT_{—}024637). As another example, the following heavy chain germline sequences found in the HCo12 HuMAb mouse are available in the accompanying Genbank accession numbers: 1-69 (NG_{—}0010109, NT_{—}024637 and BC070333), 5-51 (NG_{—}0010109 and NT_{—}024637), 4-34 (NG_{—}0010109 and NT_{—}024637), 3-30.3 (CAJ556644) and (AJ406678).

Antibody protein sequences are compared against a compiled protein sequence database using a sequence similarity searching method called Gapped BLAST (Altschul et al. (1997) Nucleic Acids Research 25:3389-3402). BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (http://vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Preferred framework sequences for use in the antibodies of the invention are those that are structurally similar to the framework sequences used by known RG-1 antibodies. The V_{H} CDR1, CDR2, and CDR3 sequences, and the V_{L} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al.).

Another type of variable region modification is to mutate amino acid residues within the V_{H} and/or V_{L} CDR1, CDR2 and/or CDR3 regions to improve one or more binding properties. Site-directed mutagenesis or PCR-mediated mutagenesis can be performed to introduce the mutation(s) and the effect on antibody binding, or other functional property of interest, can be evaluated in in vitro or in vivo assays. Preferably conservative modifications are introduced. The mutations may be amino acid substitutions, additions or deletions, but are preferably substitutions. Moreover, typically no more than one, two, three, four or five residues within a CDR region are altered.

Accordingly, in another embodiment, the antibody or antigen binding portions thereof in a conjugate of this invention comprises: (a) a V_{H} CDR1 region comprising an amino acid sequence selected from SEQ ID NOs: 1-2; (b) a V_{H} CDR2 region comprising an amino acid sequence selected from SEQ ID NOs: 3-4; (c) a V_{H} CDR3 region comprising an amino acid sequence selected from SEQ ID NOs: 5-6; (d) a V_{L} CDR1 region comprising an amino acid sequence selected from SEQ ID NOs: 7-8; (e) a V_{L} CDR2 region comprising an amino acid sequence selected from SEQ ID NOs: 9-10; and (f) a V_{L} CDR3 region comprising an amino acid sequence selected from SEQ ID NOs: 11-12; wherein at least one of the aforementioned V_{H} CDR1, CDR2, and CDR3 and V_{L} CDR1, CDR2, and CDR3 has one, two, three, four, or five (preferably one or two) amino acid substitutions, deletions or additions as compared to the referenced SEQ ID NOs.

Engineered antibodies include those in which modifications have been made to framework residues within V_{H} and/or V_{L}, typically to decrease immunogenicity. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes, also to reduce immunogenicity. This approach is also referred to as “deimmunization” and is described in US 2003/0153043 by Carr et al.

Antibodies also may be engineered to include modifications within the Fc region, typically to alter properties such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of C_{H}1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease its biological half life. More specifically, one or more amino acid mutations are introduced into the CH2-CH3 domain interface region of the Fc-hinge fragment such that the antibody has impaired Staphylococcyl protein A (SpA) binding relative to native Fe-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or C_{L} region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in WO 94/29351 by Bodmer et al.

In yet another example, the Fc region is modified to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in WO 00/42072 by Presta. Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in King et al., international application PCT/US2008/073569, which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

Such C-terminal cysteine modifications can provide a functional group for conjugation of the partner molecule, for example via a disulfide linker or a maleimide group. Conjugation of the antibody to the partner molecule in this manner allows for increased control over the specific site of attachment. Furthermore, by introducing the site of attachment at or near the C-terminus, conjugation can be optimized such that it reduces or eliminates interference with antibody-antigen binding, and allows for simplified analysis and quality control.

In still another embodiment, the antibody can be engineered to be aglycoslated (i.e., lacks glycosylation), to increase binding to the antigen. Such modifications can be accomplished by altering one or more glycosylation sites within the antibody sequence. For example, one or more amino acid substitutions can be made that result in elimination of one or more variable region framework glycosylation sites, preventing glycosylation at that site. Such an approach is described in further detail in U.S. Pat. Nos. 5,714,350 and 6,350,861 to Co et al. Additional approaches for altering glycosylation are described in U.S. Pat. No. 7,214,775 to Hanai et al., U.S. Pat. No. 6,737,056 to Presta, US 2007/0020260 to Presta, WO 2007/084926 to Dickey et al., WO 2006/089294 to Zhu et al., and WO 2007/055916 to Ravetch et al., each of which is hereby incorporated by reference in its entirety.

An antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC. Such modifications can be accomplished by expressing the antibody in a host cell with altered glycosylation machinery. Cells with altered glycosylation machinery have been described in the art and can be used as host cells in which to express recombinant antibodies to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in these cell lines lack fucose on their carbohydrates. Such cell lines were created by the targeted disruption of the FUT8 gene in CHO/DG44 cells using two replacement vectors (see US 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al. (2002) J. Biol. Chem. 277:26733-26740). WO 99/54342 by Umana et al. describes cell lines engineered to express glycoprotein-modifying glycosyl such that antibodies expressed in them exhibit increased bisecting GlcNac structures, which results in increased ADCC activity of the antibodies (see also Umana et al. (1999) Nat. Biotech. 17:176-180). Alternatively, the fucose residues of the antibody may be cleaved off using a fucosidase enzyme such as alpha-L-fucosidase removes fucosyl residues from antibodies (Tarentino et al. (1975) Biochem. 14:5516-23).

Additionally or alternatively, an antibody can be made that has an altered level of sialylation. such as described in WO 2007/084926 to Dickey et al, and WO 2007/055916 to Ravetch et al., both of which are incoporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al., J. Virology, 15(4), 882-893 (1975) and in Leibiger et al., Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51(3), 307-311 (2000).

Another contemplated modification of the antibodies used herein is pegylation. An antibody can be pegylated to increase the biological (e.g., serum) half life of the antibody. To pegylate an antibody, the antibody, or fragment thereof; typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” encompasses any of the forms of PEG that have been used to derivatize other proteins, such as mono (C1-C10) alkoxy- or aryloxypolyethylene glycol or polyethylene glycol-maleimide. In certain embodiments, the antibody to be pegylated is an aglycosylated antibody. Methods for pegylating proteins are known in the art. See for example, EP 0154316 by Nishimura et al. and EP 0401384 by Ishikawa et al.

### Antibody Physical Properties

The antibodies used in the present invention may be characterized by the various physical properties.

The antibodies may contain one or more glycosylation sites in either the V_{L} or V_{H}, which may result in it having increased immunogenicity or altered pK (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43 R-56R; Parekh et al (1985) Nature 316:452-7; Mimura et al. (2000) Mol Immunol 37:697-706). Glycosylation has been known to occur at motifs containing an N-X-S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it is preferred to have an anti-RG-1 antibody that does not contain variable region glycosylation. This can be achieved either by selecting antibodies that do not contain the glycosylation motif in the variable region or by mutating residues within the glycosylation motif using standard techniques.

In a preferred embodiment, the antibodies of the present disclosure do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect). The presence of isoaspartic acid can be measured using a reverse-phase HPLC test (iso-quant assay).

Each antibody will have a unique isoelectric point (pI), generally falling in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-mesothelin antibody that contains a pI value that falls in the normal range. This can be achieved either by selecting antibodies with a pI in the normal range or by mutating charged surface residues.

Each antibody will have a characteristic melting temperature, with a higher melting temperature indicating greater overall stability in vivo (Krishnamurthy R and Manning M C (2002) Curr Pharm Biotechnol 3:361-71). Generally, it is preferred that the T_{M1} (the temperature of initial unfolding) be greater than 60° C., preferably greater than 65° C., even more preferably greater than 70° C. The melting point of an antibody can be measured using differential scanning calorimetry (Chen et al (2003) Pharm Res 20:1952-60; Ghirlando et al (1999) Immunol Lett 68:47-52) or circular dichroism (Murray et al. (2002) J. Chromatogr Sci 40:343-9).

In a preferred embodiment, antibodies are selected that do not rapidly degrade. Fragmentation of an anti-RG-1 antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects, which can lead to the triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation can be measured by several techniques, including size-exclusion column (SEC), high performance liquid chromatography (HPLC), and light scattering.

### Methods of Engineering Antibodies

As discussed above, the anti-RG-1 antibodies having known V_{H} and V_{L} sequences can be used to create new anti-RG-1 antibodies by modifying the V_{H} and/or V_{L} sequences, or the constant region(s) attached thereto. Thus, in another aspect of the invention, the structural features of known anti-RG-1 antibodies are used to create structurally related anti-RG-1 antibodies that retain at least one functional property of the antibodies of the invention, such as binding to human RG-1. For example, one or more CDR regions of a known RG-1 antibody or mutations thereof, can be combined recombinantly with known framework regions and/or other CDRs to create additional, recombinantly-engineered, anti-RG-1 antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the known RG-1 antibody V_{H} and/or V_{K} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Mutations can be introduced randomly or selectively along all or part of an anti-RG-1 antibody coding sequence and the resulting modified anti-RG-1 antibodies can be screened for binding activity and/or other functional properties as described herein. Mutational methods have been described in the art. For example, WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

### Antibody Fragments and Antibody Mimetics

The conjugates of this invention are not limited traditional antibodies as the RG-1 binding component and may be practiced through the use of antibody fragments and antibody mimetics. A wide variety of antibody fragment and antibody mimetic technologies have now been developed and are widely known in the art.

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

Nanobodies are antibody-derived proteins that contain the unique structural and functional properties of naturally-occurring heavy-chain antibodies. These heavy-chain antibodies contain a single variable domain (VHH) and two constant domains (CH2 and CH3).

Importantly, the cloned and isolated VHH domain is a stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential.

Nanobodies combine the advantages of conventional antibodies with important features of small molecule drugs. Like conventional antibodies, Nanobodies show high target specificity and affinity and low inherent toxicity. Furthermore, Nanobodies are extremely stable, can be administered by means other than injection (see, e.g., WO 2004/041867) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety).

The Nanoclone method (see, e.g., WO 06/079372, which is herein incorporated by reference in its entirety) generates Nanobodies against a desired target, based on automated high-throughout selection of B-cells and could be used in the context of the instant invention.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

Affibody molecules are affinity proteins based on a 58-amino acid residue protein domain derived from a three helix bundle IgG-binding domain of staphylococcal protein A. This domain has been used as a scaffold for the construction of combinatorial phagemid libraries, from which Affibody variants targeting the desired molecules can be selected using phage display technology (Nord et al., Nat Biotechnol 1997; 15:772-7; Ronmark et al., Eur J Biochem 2002; 269:2647-55). The simple, robust structure and low molecular weight (6 kDa) of Affibody molecules makes them suitable for a wide variety of applications, such as detection reagents and inhibitors of receptor interactions. Further details on Affibodies are found in U.S. Pat. No. 5,831,012 which is incorporated by reference in its entirety. Labelled Affibodies may also be useful in imaging applications for determining abundance of isoforms.

DARPins (Designed Ankyrin Repeat Proteins) embody DRP (Designed Repeat Protein) antibody mimetic technology that exploits the binding abilities of non-antibody polypeptides. Repeat proteins, such as ankyrin and leucine-rich repeat proteins, are ubiquitous binding molecules that, unlike antibodies, occur intra- and extracellularly. Their unique modular architecture features repeating structural units (repeats) that stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains Additional information regarding DARPins and other DRP technologies can be found in US 2004/0132028 and WO 02/20565, both of which are incorporated by reference.

Anticalins are another antibody mimetic technology. In this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids, which is marginally larger than a single immunoglobulin domain.

Lipocalins can be cloned and their loops subjected to engineering to create Anticalins. Libraries of structurally diverse Anticalins have been generated and Anticalin display allows the selection and screening of binding function, followed by the expression and production of soluble protein for further analysis in prokaryotic or eukaryotic systems. Studies have demonstrated that Anticalins can be developed that are specific for virtually any human target protein and binding affinities in the nanomolar or higher range can be obtained. Additional information regarding Anticalins can be found in U.S. Pat. No. 7,250,297 and WO 99/16873, both of which are hereby incorporated by reference in their entirety.

Avimers are another type of antibody mimetic technology useful in the context of the instant invention. Avimers are evolved from a large family of human extracellular receptor domains by in vitro exon shuffling and phage display, generating multidomain proteins with binding and inhibitory properties Linking multiple independent binding domains has been shown to create avidity and results in improved affinity and specificity compared to conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets. Additional information regarding Avimers can be found in US 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that can be used in the context of the instant invention. Versabodies are small proteins of 3-5 kDa with >15% cysteines, which form a high disulfide density scaffold replacing the hydrophobic core that typical proteins have. This replacement results in a protein that is smaller, is more hydrophilic (i.e., less prone to aggregation and non-specific binding), is more resistant to proteases and heat, and has a lower density of T-cell epitopes, because the residues that contribute most to MHC presentation are hydrophobic. these properties are well-known to affect immunogenicity, and together they are expected to cause a large decrease in immunogenicity.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable and offer extended shelf-life. Additional information regarding Versabodies can be found in US 2007/0191272, which is hereby incorporated by reference in its entirety.

The above descriptions of antibody fragment and mimetic technologies is not intended to be comprehensive. A variety of additional technologies including alternative polypeptide-based technologies, such as fusions of complementarity determining regions as outlined in Qui et al., Nature Biotechnology, 25(8) 921-929 (2007), as well as nucleic acid-based technologies, such as the RNA aptamer technologies described in U.S. Pat. Nos. 5,789,157; 5,864,026; 5,712,375; 5,763,566; 6,013,443; 6,376,474; 6,613,526; 6,114,120; 6,261,774; and 6,387,620; all of which are hereby incorporated by reference, could be used in the context of the instant invention.

### Nucleic Acid Molecules Encoding Antibodies of the Invention

Another aspect of the invention pertains to nucleic acid molecules that encode the antibodies of the invention. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

The nucleic acids can be obtained using standard molecular biology techniques. For antibodies expressed by hybridomas (e.g., hybridomas prepared from transgenic mice carrying human immunoglobulin genes as described below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids are those encoding the V_{H} and V_{L} sequences of the 19G9 or 34E1 monoclonal antibodies. DNA sequences encoding the V_{H} sequences of 19G9 and 34E1 are shown in SEQ ID NOs: 17-18, respectively. DNA sequences encoding the V_{L} sequences of 19G9 and 34E1 are shown in SEQ ID NOs: 19-20, respectively.

Once DNA fragments encoding V_{H} and V_{L} segments are obtained, they can be manipulated by standard recombinant DNA techniques, for example to convert the variable region genes to full-length antibody chain genes, to Fab fragment genes, or to a scFv gene. In these manipulations, a V_{L}- or V_{H}-encoding DNA fragment is operatively linked to another DNA fragment encoding another protein, such as an antibody constant region or a flexible linker. The term “operatively linked,” as used in this context, means that the two DNA fragments are joined such that the amino acid sequences encoded by them remain in-frame.

The isolated DNA encoding the V_{H} region can be converted to a full-length heavy chain gene by operatively linking the V_{H}-encoding DNA to another DNA molecule encoding heavy chain constant regions C_{H}1, C_{H}2 and C_{H}3. The sequences of human heavy chain constant region genes are known in the art (see e.g., Kabat '3242) and DNA fragments encompassing them can be obtained by PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to DNA encoding only the heavy chain C_{H}1 constant region.

The isolated DNA encoding the V_{L} region can be converted to a full-length light chain gene (as well as a Fab light chain gene) by operatively linking the V_{L}-encoding DNA to another DNA molecule encoding the light chain constant region C_{L}. The sequences of human light chain constant region genes are known in the art (see e.g., Kabat '3242) and DNA fragments encompassing them can be obtained by PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies

Monoclonal antibodies (mAbs) for use in the present invention can be produced by a variety of techniques, including conventional monoclonal antibody methodology, e.g., the somatic cell hybridization technique of Kohler and Milstein (1975) Nature 256: 495. Although somatic cell hybridization procedures are preferred, in principle other techniques can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

Preferably, the antibodies of the invention are human monoclonal antibodies. Such human monoclonal antibodies directed against RG-1 can be generated using transgenic or transchromosomic mice carrying parts of the human immune system rather than the mouse system. These transgenic and transchromosomic mice include mice referred to herein as mice of HuMAb Mouse® and KM Mouse® types or strains, respectively, and are collectively referred to herein as “human Ig mice.”

The HuMAb Mouse® strain (Medarex®, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg et al. (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg et al. (1994), supra; reviewed in Lonberg (1994) Handbook of Experimental Pharmacology 113:49-101; Lonberg and Huszar (1995) Intern. Rev. Immunol. 13: 65-93, and Harding and Lonberg (1995) Ann. N.Y. Acad. Sci. 764:536-546). Preparation and use of mice of the HuMAb Mouse® strain, and the genomic modifications carried by such mice, is further described in Taylor et al. (1992) Nucleic Acids Research 20:6287-6295; Chen et al. (1993) International Immunology 5: 647-656; Tuaillon et al. (1993) Proc. Natl. Acad. Sci. USA 90:3720-3724; Choi et al. (1993) Nature Genetics 4:117-123; Chen et al. (1993) EMBO J. 12: 82**30; Tuaillon et al. (1994) J. Immunol. 152:2912-2920; Taylor et al. (1994) International Immunology 6: 579-591; and Fishwild et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; WO 92/03918, WO 93/12227, WO 94/25585, WO 97/13852, WO 98/24884 and WO 99/45962, all to Lonberg and Kay; and WO 01/14424 to Korman et al.

In another embodiment, human antibodies can be generated using a mouse carrying human immunoglobulin sequences on transgenes and transchomosomes, e.g. a human heavy chain transgene and a human light chain transchromosome. Such a mouse is referred to herein as being of the “KM Mouse®” type and is described in detail in WO 02/43478 to Ishida et al.

Still further, alternative transgenic animal systems expressing human immunoglobulin genes are available in the art and can be used to raise anti-RG-1 antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-RG-1 antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and WO 2002/092812 and can be used to generate anti-RG-1 antibodies of the invention.

Human monoclonal antibodies of the invention can also be prepared using phage display methods for screening libraries of human immunoglobulin genes. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Immunization of Human Ig Mice

When human Ig mice are used to generate human antibodies, they can be immunized with a purified or enriched preparation of RG-1 antigen and/or recombinant RG-1, or cells expressing RG-1, or an RG-1 fusion protein, as described by Lonberg et al. (1994) Nature 368(6474): 856-859; Fishwild et al. (1996) Nature Biotechnology 14: 845-851; and WO 98/24884 and WO 01/14424. Preferably, the mice will be 6-16 weeks of age upon the first infusion. For example, a purified or recombinant preparation (5-50 μg) of RG-1 antigen can be used to immunize the human Ig mice intraperitoneally.

Cumulative experience with various antigens has shown that the transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immuno-genic. The immune response can be monitored over the course of the immunization protocol with plasma samples being obtained by retroorbital bleeds. The plasma can be screened by ELISA and mice with sufficient titers of anti-RG-1 human immunoglobulin can be used for fusions. Mice can be boosted intravenously with antigen 3 days before sacrifice and removal of the spleen. It is expected that 2-3 fusions for each immunization may be needed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo12). Alternatively or additionally, the KM Mouse® strain can be used.

### Generation of Hybridomas Producing Human Monoclonal Antibodies

To generate hybridomas producing human monoclonal antibodies, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed, usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using an extinction coefficient of 1.43. The monoclonal antibodies can be aliquoted and stored at −80° C.

### Generation of Transfectomas Producing Monoclonal Antibodies

Antibodies for use in the invention also can be produced in a host cell transfectoma using, for example, a combination of recombinant DNA techniques and gene transfection methods (e.g., Morrison, S. (1985) Science 229:1202).

To express the antibodies, or antibody fragments thereof, DNAs encoding partial or full-length light and heavy chains can be obtained by standard techniques (e.g., PCR amplification or cDNA cloning using a hybridoma that expresses the antibody of interest) and the DNAs can be inserted into expression vectors such that the genes are operatively linked to transcriptional and translational control sequences. The term “operatively linked” means that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The V_{L}s and V_{H}S of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as those regulating replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13).

Preferred mammalian host cells for expressing the recombinant antibodies of the invention include Chinese Hamster Ovary (CHO cells) (including dhfr⁻ CHO cells, described in Urlaub and Chasin, (1980) Proc. Natl. Acad. Sci. USA 77:4216-4220, used with a DHFR selectable marker, e.g., as described in Kaufman and Sharp (1982) J. Mol. Biol. 159:601-621), NSO myeloma cells, COS cells and SP2 cells. For use with NSO myeloma cells, a preferred expression system is the GS gene expression system disclosed in WO 87/04462 (to Wilson), WO 89/01036 (to Bebbington) and EP 338,841 (to Bebbington). When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

### Characterization of Antibody Binding to Antigen

Antibodies can be tested for binding to RG-1 by standard ELISA. Briefly, microtiter plates are coated with purified RG-1 at 0.25 μg/ml in PBS, and then blocked with 5% bovine serum albumin in PBS. Dilutions of antibody (e.g., dilutions of plasma from RG-1-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with RG-1 immunogen. Hybridomas that bind with high avidity to RG-1 are subcloned and further characterized. One clone from each hybridoma, which retains the reactivity of the parent cells (by ELISA), can be chosen for making a 5-10 vial cell bank stored at −140° C., and for antibody purification.

To purify anti-RG-1 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if the selected anti-RG-1 monoclonal antibodies bind to unique epitopes, an antibody can be biotinylated using commercially available reagents (Pierce, Rockford, Ill.). Competition studies using unlabeled monoclonal antibodies and biotinylated monoclonal antibodies can be performed using RG-1 coated-ELISA plates. Biotinylated mAb binding can be detected with a strepavidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-RG-1 human IgGs can be further tested for reactivity with RG-1 antigen by Western blotting. Briefly, RG-1 is prepared and subjected to sodium dodecyl sulfate polyacrylamide gel electrophoresis. Then, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The binding specificity of an antibody of the invention may also be determined by monitoring binding of the antibody to cells expressing RG-1, for example by flow cytometry. Typically, a cell line, such as a CHO cell line, may be transfected with an expression vector encoding a transmembrane form of RG-1. The transfected protein may comprise a tag, such as a myc-tag, preferably at the N-terminus, for detection using an antibody to the tag. Binding of an antibody of the invention to RG-1 may be determined by incubating the transfected cells with the antibody, and detecting bound antibody. Binding of an antibody to the tag on the transfected protein may be used as a positive control.

The specificity of an antibody of the invention for RG-1 may be further studied by determining whether or not the antibody binds to other proteins, such as PROTEIN Y or RG-1 using the same methods by which binding to RG-1 is determined.

### Bispecific Molecules

The antibody portion of a conjugate can be a bispecific molecule. An anti-RG-1 antibody, or a fragment thereof, can be derivatized with or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule, an antibody can be functionally linked (e.g., by chemical coupling, genetic fusion, noncovalent association or otherwise) to one or more other binding molecules, such as another antibody, antibody fragment, peptide or binding mimetic, such that a bispecific molecule results.

The bispecific molecules comprise at least one first binding specificity for RG-1 and a second binding specificity for a second target epitope such as an Fc receptor, e.g., human FcγRI (CD64) or a human Fcα receptor (CD89). Such bispecific molecules capable of binding both to FcγR or FcαR expressing effector cells (e.g., monocytes, macrophages or polymorphonuclear cells (PMNs)) and to cells expressing RG-1. These bispecific molecules target RG-1 expressing cells to effector cell and trigger Fc receptor-mediated effector cell activities, such as phagocytosis of an RG-1 expressing cells, antibody dependent cell-mediated cytotoxicity (ADCC), cytokine release, or generation of superoxide anion.

A bispecific molecule can in fact be multispecific, i.e., it can further include a third binding specificity, in addition to an anti-Fc binding specificity and an anti-RG-1 binding specificity. In one embodiment, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind to an Fc receptor or a target cell antigen or, alternatively, to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one embodiment, the bispecific molecules of the invention comprise as a binding specificity at least one antibody, or an antibody fragment thereof, including, e.g., an Fab, Fab′, F(ab)₂, Fv, Fd, dAb or a single chain Fv. The antibody may also be a light chain or heavy chain dimer, or any minimal fragment thereof such as a Fv or a single chain construct as described in U.S. Pat. No. 4,946,778 to Ladner et al., the contents of which is expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII (CD32), and FcγRIII (CD 16). In one preferred embodiment, the Fcγ receptor a human high affinity FcγRI. The human FcγRI is a 72 kDa molecule, which shows high affinity for monomeric IgG (10⁸-10⁹ M⁻¹).

The production and characterization of anti-Fcγ monoclonal antibodies are described in WO 88/00052 and in U.S. Pat. No. 4,954,617 to Fanger et al., which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano et al. (1995) J. Immunol. 155 (10): 4996-5002 and WO 94/10332 to Tempest et al. The H22 antibody producing cell line is deposited at the American Type Culture Collection under the designation HA022CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/ma-crophages, eosinophilic and neutrophilic granulocytes, but not on non-effector cell populations. FcαRI has medium affinity (≈5×10⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); and (4) mediate enhanced antigen presentation of antigens, including self-antigens, targeted to them.

While human monoclonal antibodies are preferred, other antibodies which can be employed in the bispecific molecules of the invention are murine, chimeric and humanized monoclonal antibodies.

Bispecific molecules can be prepared by conjugating the constituent binding specificities, e.g., the anti-FcR and anti-RG-1 binding specificities, using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al. (1984) J. Exp. Med. 160:1686; Liu et al. (1985) Proc. Natl. Acad. Sci. USA 82:8648). Other methods include those described in Paulus (1985) Behring Ins. Mitt. No. 78, 118-132; Brennan et al. (1985) Science 229:8**3, and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858, all of which are expressly incorporated herein by reference.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of [symbol] counter or a scintillation counter or by autoradiography.

### Conjugates

In conjugates of this invention, the partner molecule is conjugated to an antibody by a chemical linker (sometimes referred to herein simply as “linker”). The partner molecule can be a therapeutic agent or a marker. The therapeutic agent can be, for example, a cytotoxin, a non-cytotoxic drug (e.g., an immunosuppressant), a radioactive agent, another antibody, or an enzyme. Preferably, the partner molecule is a cytotoxin. The marker can be any label that generates a detectable signal, such as a radiolabel, a fluorescent label, or an enzyme that catalyzes a detectable modification to a substrate. The antibody serves a targeting function: by binding to a target tissue or cell where its antigen is found, the antibody steers the conjugate to the target tissue or cell. There, the linker is cleaved, releasing the partner molecule to perform its desired biological function.

The ratio of partner molecules attached to an antibody can vary, depending on factors such as the amount of partner molecule employed during conjugation reaction and the experimental conditions. Preferably, the ratio of partner molecules to antibody is between 1 and 3, more preferably between 1 and 1.5. Those skilled in the art will appreciate that, while each individual molecule of antibody Z is conjugated to an integer number of partner molecules, a preparation of the conjugate may analyze for a non-integer ratio of partner molecules to antibody, reflecting a statistical average.

### Linkers

In some embodiments, the linker is a peptidyl linker, depicted herein as (L⁴)ₚ-F-(L¹)ₘ. Other linkers include hydrazine and disulfide linkers, depicted herein as (L⁴)ₚ-H-(L¹)ₘ, and (L⁴)ₚ-J-(L¹)ₘ, respectively. F, H, and J are peptidyl, hydrazine, and disulfide moieties, respectively, that are cleavable to release the partner molecule from the antibody, while L¹ and L⁴ are linker groups. F, H, J, L¹, and L⁴ are more fully defined hereinbelow, along with the subscripts p and m. The preparation and use of these and other linkers are described in WO 2005/112919, the disclosure of which is incorporated herein by reference.

The use of peptidyl and other linkers in antibody-partner conjugates is described in US 2006/0004081; 2006/0024317; 2006/0247295; 6,989,452; 7,087,600; and 7,129,261; WO 2007/051081; 2007/038658; 2007/059404; and 2007/089100; all of which are incorporated herein by reference.

Additional linkers are described in U.S. Pat. Nos. 6,214,345; 2003/0096743; and 2003/0130189; de Groot et al., J. Med. Chem. 42, 5277 (1999); de Groot et al. J. Org. Chem. 43, 3093 (2000); de Groot et al., J. Med. Chem. 66, 8815, (2001); WO 02/083180; Carl et al., J. Med. Chem. Lett. 24, 479, (1981); Dubowchik et al., Bioorg & Med. Chem. Lett. 8, 3347 (1998), the disclosures of which are incorporated herein by reference.

In addition to connecting the antibody and the partner molecule, a linker can impart stability to the partner molecule, reduce its in vivo toxicity, or otherwise favorably affect its pharmacokinetics, bioavailability and/or pharmacodynamics. It is generally preferred that the linker is cleaved, releasing the partner molecule, once the conjugate is delivered to its site of action. Also preferably, the linkers are traceless, such that once cleaved, no trace of the linker's presence remains.

In another embodiment, the linkers are characterized by their ability to be cleaved at a site in or near a target cell such as at the site of therapeutic action or marker activity of the partner molecule. Such cleavage can be enzymatic in nature. This feature aids in reducing systemic activation of the partner molecule, reducing toxicity and systemic side effects. Preferred cleavable groups for enzymatic cleavage include peptide bonds, ester linkages, and disulfide linkages, such as the aforementioned F, H, and J moieties. In other embodiments, the linkers are sensitive to pH and are cleaved through changes in pH.

An important aspect is the ability to control the speed with which the linkers cleave. Often a linker that cleaves quickly is desired. In some embodiments, however, a linker that cleaves more slowly may be preferred. For example, in a sustained release formulation or in a formulation with both a quick release and a slow release component, it may be useful to provide a linker which cleaves more slowly. The aforecited WO 2005/112919 discloses hydrazine linkers that can be designed to cleave at a range of speeds, from very fast to very slow.

The linkers can also serve to stabilize the partner molecule against degradation while the conjugate is in circulation, before it reaches the target tissue or cell. This is a significant benefit since it prolongates the circulation half-life of the partner molecule. The linker also serves to attenuate the activity of the partner molecule so that the conjugate is relatively benign while in circulation but the partner molecule has the desired effect—for example is cytotoxic—after activation at the desired site of action. For therapeutic agent conjugates, this feature of the linker serves to improve the therapeutic index of the agent.

In addition to the cleavable peptide, hydrazine, or disulfide groups F, H, or J, respectively, one or more linker groups L¹ are optionally introduced between the partner molecule and F, H, or J, as the case may be. These linker groups L¹ may also be described as spacer groups and contain at least two functional groups. Depending on the value of the subscript m (i.e., the number of L¹ groups present) and the location of a particular group L¹, a chemical functionality of a group L¹ can bond to a chemical functionality of the partner molecule, of F, H or J, as the case may be, or of another linker group L¹ (if more than one L¹ is present). Examples of suitable chemical functionalities for spacer groups L¹ include hydroxy, mercapto, carbonyl, carboxy, amino, ketone, aldehyde, and mercapto groups.

The linkers L¹ can be a substituted or unsubstituted alkyl, substituted or unsubstituted aryl, substituted or unsubstituted heteroaryl or substituted or unsubstituted heteroalkyl group. In one embodiment, the alkyl or aryl groups may comprise between 1 and 20 carbon atoms. They may also comprise a polyethylene glycol moiety.

Exemplary groups L¹ include, for example, 6-aminohexanol, 6-mercaptohexanol, 10-hydroxydecanoic acid, glycine and other amino acids, 1,6-hexanediol, β-alanine, 2-aminoethanol, cysteamine (2-aminoethanethiol), 5-aminopentanoic acid, 6-aminohexanoic acid, 3-maleimidobenzoic acid, phthalide, α-substituted phthalides, the carbonyl group, aminal esters, nucleic acids, peptides and the like.

One function of the groups L¹ is to provide spatial separation between F, H or J, as the case may be, and the partner molecule, lest the latter interfere (e.g., via steric or electronic effects) with cleavage chemistry at F, H, or J. The groups L¹ also can serve to introduce additional molecular mass and chemical functionality into conjugate. Generally, the additional mass and functionality affects the serum half-life and other properties of the conjugate. Thus, through careful selection of spacer groups, conjugates with a range of serum half-lives can be produced. Optionally, one or more linkers L¹ can be a self-immolative group, as described hereinbelow.

The subscript m is an integer selected from 0, 1, 2, 3, 4, 5, and 6. When multiple L¹ groups are present, they can be the same or different.

L⁴ is a linker moiety that provides spatial separation between F, H, or J, as the case may be, and the antibody, lest F, H, or J interfere with the antigen binding by the antibody or the antibody interfere with the cleavage chemistry at F, H, or J. Preferably, L⁴ imparts increased solubility or decreased aggregation properties to conjugates utilizing a linker that contains the moiety or modifies the hydrolysis rate of the conjugate. As in the case of L¹, L⁴ optionally is a self immolative group. In one embodiment, L⁴ is substituted alkyl, unsubstituted alkyl, substituted aryl, unsubstituted aryl, substituted heteroalkyl, or unsubstituted heteroalkyl, any of which may be straight, branched, or cyclic. The substitutions can be, for example, a lower (C₁-C₆) alkyl, alkoxy, alkylthio, alkylamino, or dialkylamino. In certain embodiments, L⁴ comprises a non-cyclic moiety. In another embodiment, L⁴ comprises a positively or negatively charged amino acid polymer, such as polylysine or polyarginine. L⁴ can comprise a polymer such as a polyethylene glycol moiety. Additionally, L⁴ can comprise, for example, both a polymer component and a small molecule moiety.

In a preferred embodiment, L⁴ comprises a polyethylene glycol (PEG) moiety. The PEG portion of L⁴ may be between 1 and 50 units long. Preferably, the PEG will have 1-12 repeat units, more preferably 3-12 repeat units, more preferably 2-6 repeat units, or even more preferably 3-5 repeat units and most preferably 4 repeat units. L⁴ may consist solely of the PEG moiety, or it may also contain an additional substituted or unsubstituted alkyl or heteroalkyl. It is useful to combine PEG as part of the L⁴ moiety to enhance the water solubility of the complex. Additionally, the PEG moiety reduces the degree of aggregation that may occur during the conjugation of the drug to the antibody.

The subscript p is 0 or 1; that is, the presence of L⁴ is optional. Where present, L⁴ has at least two functional groups, with one functional group binding to a chemical functionality in F, H, or J, as the case may be, and the other functional group binding to the antibody. Examples of suitable chemical functionalities of groups L⁴ include hydroxy, mercapto, carbonyl, carboxy, amino, ketone, aldehyde, and mercapto groups. As antibodies typically are conjugated via sulfhydryl groups (e.g., from unoxidized cysteine residues, the addition of sulfhydryl-containing extensions to lysine residues with iminothiolane, or the reduction of disulfide bridges), amino groups (e.g., from lysine residues), aldehyde groups (e.g., from oxidation of glycoside side chains), or hydroxyl groups (e.g., from serine residues), preferred chemical functionalities for attachment to the antibody are those reactive with the foregoing groups, examples being maleimide, sulfhydryl, aldehyde, hydrazine, semicarbazide, and carboxyl groups. The combination of a sulfhydryl group on the antibody and a maleimide group on L⁴ is preferred.

In some embodiments, L⁴ comprises

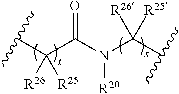

directly attached to the N-terminus of (AA¹)_{c}. R²⁰ is a member selected from H, substituted or unsubstituted alkyl, substituted or unsubstituted heteroalkyl, and acyl. Each R²⁵, R^{25′}, R²⁶, and R^{26′} is independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl; and s and t are independently integers from 1 to 6. Preferably, R²⁰, R²⁵, R^{25′}, R²⁶ and R^{26′} are hydrophobic. In some embodiments, R²⁰ is H or alkyl (preferably, unsubstituted lower alkyl). In some embodiments, R²⁵, R^{25′}, R²⁶ and R^{26′} are independently H or alkyl (preferably, unsubstituted C¹ to C⁴ alkyl). In some embodiments, R²⁵, R^{25′}, R²⁶ and R^{26′} are all H. In some embodiments, t is 1 and s is 1 or 2.

### Peptide Linkers (F)

As discussed above, the peptidyl linkers of the invention can be represented by the general formula: (L⁴)ₚ-F-(L¹)ₘ, wherein F represents the portion comprising the peptidyl moiety. In one embodiment, the F portion comprises an optional additional self-immolative linker L² and a carbonyl group, corresponding to a conjugate of formula (a):

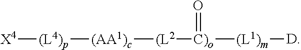

In this embodiment, L¹, L⁴, p, and m are as defined above. X⁴ is an antibody and D is a partner molecule. The subscript o is 0 or 1 and L², if present, represents a self-immolative linker AA¹ represents one or more natural amino acids, and/or unnatural α-amino acids; c is an integer from 1 and 20. In some embodiments, c is in the range of 2 to 5 or c is 2 or 3.

In formula (a), AA¹ is linked, at its amino terminus, either directly to L⁴ or, when L⁴ is absent, directly to X⁴. In some embodiments, when L⁴ is present, L⁴ does not comprise a carboxylic acyl group directly attached to the N-terminus of (AA¹)_{c}.

In another embodiment, the F portion comprises an amino group and an optional spacer group L³ and L¹ is absent (i.e., m is 0), corresponding to a conjugate of formula (b):

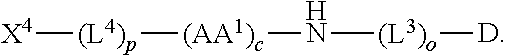

In this embodiment, X⁴, D, L⁴, AA¹, c, and p are as defined above. The subscript o is 0 or 1. L³, if present, is a spacer group comprising a primary or secondary amine or a carboxyl functional group, and either the amine of L³ forms an amide bond with a pendant carboxyl functional group of D or the carboxyl of L³ forms an amide bond with a pendant amine functional group of D.

### Self-Immolative Linkers

A self-immolative linker is a bifunctional chemical moiety which is capable of covalently linking together two spaced chemical moieties into a normally stable tripartate molecule, releasing one of said spaced chemical moieties from the tripartate molecule by means of enzymatic cleavage; and following said enzymatic cleavage, spontaneously cleaving from the remainder of the molecule to release the other of said spaced chemical moieties. In accordance with the present invention, the self-immolative spacer is covalently linked at one of its ends to the peptide moiety and covalently linked at its other end to the chemically reactive site of the drug moiety whose derivatization inhibits pharmacological activity, so as to space and covalently link together the peptide moiety and the drug moiety into a tripartate molecule which is stable and pharmacologically inactive in the absence of the target enzyme, but which is enzymatically cleavable by such target enzyme at the bond covalently linking the spacer moiety and the peptide moiety to thereby effect release of the peptide moiety from the tripartate molecule. Such enzymatic cleavage, in turn, will activate the self-immolating character of the spacer moiety and initiate spontaneous cleavage of the bond covalently linking the spacer moiety to the drug moiety, to thereby effect release of the drug in pharmacologically active form. See, for example, Carl et al., J. Med. Chem., 24 (3), 479-480 (1981); Carl et al., WO 81/01145 (1981); Told et al., J. Org. Chem. 67, 1866-1872 (2002); Boyd et al., WO 2005/112919; and Boyd et al., WO 2007/038658, the disclosures of which are incorporated herein by reference.

One particularly preferred self-immolative spacer may be represented by the formula (c):

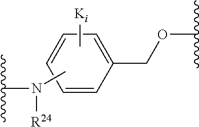

The aromatic ring of the aminobenzyl group may be substituted with one or more “K” groups. A “K” group is a substituent on the aromatic ring that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Each K is independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², N²¹COR²², OCONR²¹R²², OCOR²¹, and OR²¹, wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl. Exemplary K substituents include, but are not limited to, F, Cl, Br, I, NO₂, OH, OCH₃, NHCOCH₃, N(CH₃)₂, NHCOCF₃ and methyl. For “Kₛ”, i is an integer of 0, 1, 2, 3, or 4. In one preferred embodiment, i is 0.

The ether oxygen atom of the above structure is connected to a carbonyl group (not shown). The line from the NR²⁴ functionality into the aromatic ring indicates that the amine functionality may be bonded to any of the five carbons that both form the ring and are not substituted by the —CH₂—O— group. Preferably, the NR²⁴ functionality of X is covalently bound to the aromatic ring at the para position relative to the —CH₂—O— group. R²⁴ is a member selected from the group consisting of H, substituted alkyl, unsubstituted alkyl, substituted heteroalkyl, and unsubstituted heteroalkyl. In a specific embodiment, R²⁴ is hydrogen.

In one embodiment, the invention provides a peptide linker of formula (a) above, wherein F comprises the structure:

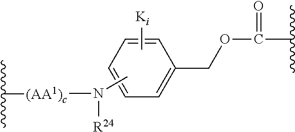

where R²⁴, AA¹, K, i, and c are as defined above.

In another embodiment, the peptide linker of formula (a) above comprises a —F-(L¹)ₘ′- that comprises the structure:

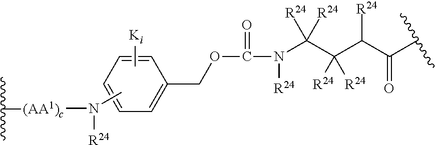

where R²⁴, AA¹, K, i, and c are as defined above.

In some embodiments, a self-immolative spacer L¹ or L² includes

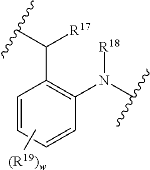

where each R¹⁷, R¹⁸, and R¹⁹ is independently selected from H, substituted or unsubstituted alkyl, substituted or unsubstituted heteroalkyl and substituted or unsubstituted aryl, and w is an integer from 0 to 4. In some embodiments, R¹⁷ and R¹⁸ are independently H or alkyl (preferably, unsubstituted C₁-C₄ alkyl). Preferably, R¹⁷ and R¹⁸ are C₁₋₄ alkyl, such as methyl or ethyl. In some embodiments, w is 0. It has been found experimentally that this particular self-immolative spacer cyclizes relatively quickly.

In some embodiments, L¹ or L² includes

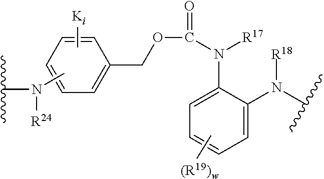

where R¹⁷, R¹⁸, R¹⁹, R²⁴, and K are as defined above.

### Spacer Groups

The spacer group L³ is characterized by comprises a primary or secondary amine or a carboxyl functional group, and either the amine of L³ forms an amide bond with a pendant carboxyl functional group of D or the carboxyl of L³ forms an amide bond with a pendant amine functional group of D. L³ can be selected from the group consisting of substituted or unsubstituted alkyl, substituted or unsubstituted heteroalkyl, substituted or unsubstituted aryl, substituted or unsubstituted heteroaryl, or substituted or unsubstituted heterocycloalkyl. In a preferred embodiment, L³ comprises an aromatic group. More preferably, L³ comprises a benzoic acid group, an aniline group or indole group. Non-limiting examples of structures that can serve as an -L³-NH— spacer include the following structures:

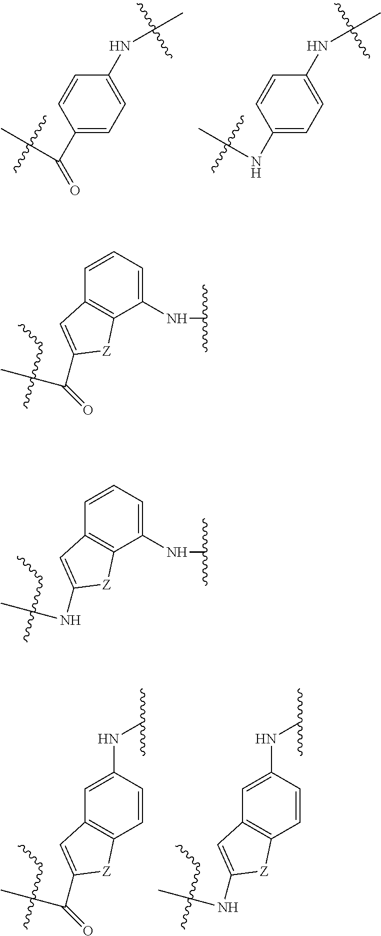

where Z is a member selected from O, S and NR²³, and where R²³ is a member selected from H, substituted or unsubstituted alkyl, substituted or unsubstituted heteroalkyl, and acyl.

Upon cleavage of the linker of the invention containing L³, the L³ moiety remains attached to the drug, D. Accordingly, the L³ moiety is chosen such that its attachment to D does not significantly alter the activity of D. In another embodiment, a portion of the drug D itself functions as the L³ spacer. For example, in one embodiment, the drug, D, is a duocarmycin derivative in which a portion of the drug functions as the L³ spacer. Non-limiting examples of such embodiments include those in which NH₂-(L³)-D has a structure selected from the group consisting of:

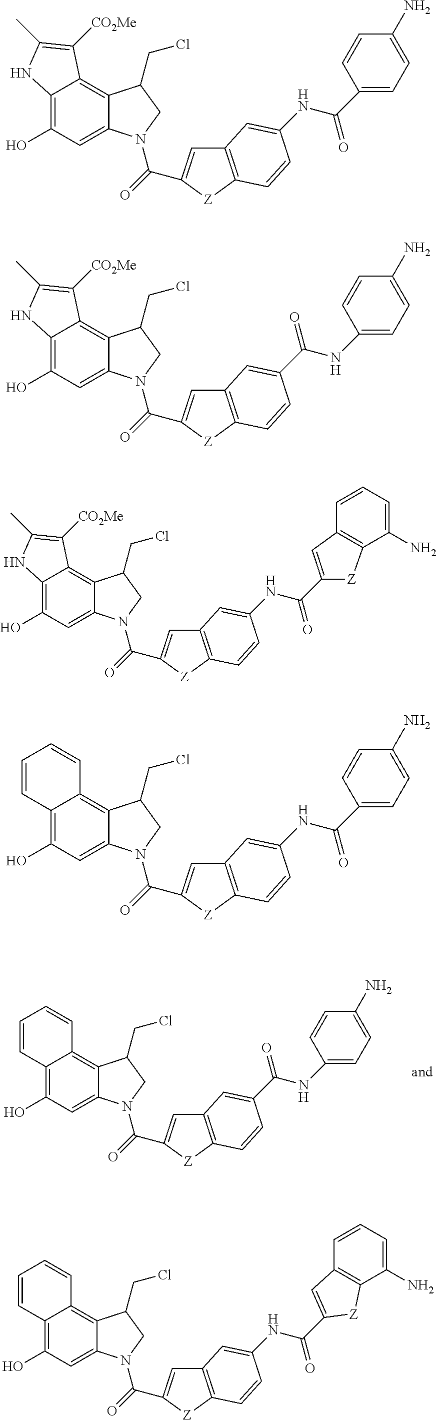

where Z is O, S or NR²³, where R²³ is H, substituted or unsubstituted alkyl, substituted or unsubstituted heteroalkyl, or acyl; and the NH₂ group on each structure reacts with (AA¹)_{c} to form -(AA¹)_{c}-NH—.

### Peptide Sequence (AA¹)_{c}

The group AA¹ represents a single amino acid or a plurality of amino acids joined together by amide bonds. The amino acids may be natural amino acids and/or unnatural α-amino acids. They may be in the L or the D configuration. In one embodiment, at least three different amino acids are used. In another embodiment, only two amino acids are used.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproiine, γ-carboxyglutamate, citrulline, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. One amino acid that may be used in particular is citrulline, which is a precursor to arginine and is involved in the formation of urea in the liver. Amino acid mimetics refers to chemical compounds that have a structure that is different from the general chemical structure of an amino acid, but functions in a manner similar to a naturally occurring amino acid. The term “unnatural amino acid” is intended to represent the “D” stereochemical form of the twenty naturally occurring amino acids described above. It is further understood that the term unnatural amino acid includes homologues of the natural amino acids, and synthetically modified forms of the natural amino acids. The synthetically modified forms include, but are not limited to, amino acids having alkylene chains shortened or lengthened by up to two carbon atoms, amino acids comprising optionally substituted aryl groups, and amino acids comprised halogenated groups, preferably halogenated alkyl and aryl groups. When attached to a linker or conjugate of the invention, the amino acid is in the form of an “amino acid side chain”, where the carboxylic acid group of the amino acid has been replaced with a keto (C(O)) group. Thus, for example, an alanine side chain is —C(O)—CH(NH₂)—CH₃, and so forth.

The peptide sequence (AA¹)_{c} is functionally the amidification residue of a single amino acid (when c=1) or a plurality of amino acids joined together by amide bonds. The peptide sequence (AA¹)_{c} preferably is selected for enzyme-catalyzed cleavage by an enzyme in a location of interest in a biological system. For example, for conjugates that are targeted to but not internalized by a cell, a peptide is chosen that is cleaved by a protease that in in the extracellular matrix, e.g., a protease released by nearby dying cells or a tumor-associated protease, such that the peptide is cleaved extracellularly. For conjugates that are designed for internalization by a cell, the sequence (AA¹)_{c} preferably is selected for cleavage by an endosomal or lysosomal protease. The number of amino acids within the peptide can range from 1 to 20; but more preferably there will be 1-8 amino acids, 1-6 amino acids or 1, 2, 3 or 4 amino acids comprising (AA¹)_{c}. Peptide sequences that are susceptible to cleavage by specific enzymes or classes of enzymes are well known in the art.

Preferably, (AA¹)_{c} contains an amino acid sequence (“cleavage recognition sequence”) that is a cleavage site by the protease. Many protease cleavage sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); Bouvier et al. Meth. Enzymol. 248: 614 (1995), Hardy et al., in Amyloid Protein Precursor in Development, Aging, and Alzheimer's Disease, ed. Masters et al. pp. 190-198 (1994).

The peptide typically includes 3-12 (or more) amino acids. The selection of particular amino acids will depend, at least in part, on the enzyme to be used for cleaving the peptide, as well as, the stability of the peptide in vivo. One example of a suitable cleavable peptide is β-Ala-Leu-Ala-Leu (SEQ ID NO: 27). This can be combined with a stabilizing group to form succinyl-β-Ala-Leu-Ala-Leu (SEQ ID NO: 30). Other examples of suitable cleavable peptides are provided in the references cited below. Alternatively, linkers comprising a single amino acid residue can be used, as disclosed in WO 2008/103693, the disclosure of which is incorporated herein by reference.

In a preferred embodiment, the peptide sequence (AA¹)_{c} is chosen based on its ability to be cleaved by a lysosomal proteases, examples of which include cathepsins B, C, D, H, L and S. Preferably, the peptide sequence (AA¹)_{c} is capable of being cleaved by cathepsin B in vitro. Though cathepsin B is a lysosomal proteaste, it is believed that a certain concentration of it is found in the extracellular matrix surrounding tumor tissues.

In another embodiment, the peptide sequence (AA¹)_{c} is chosen based on its ability to be cleaved by a tumor-associated protease, such as a protease found extracellularly in the vicinity of tumor cells, examples of which include thimet oligopeptidase (TOP) and CD10. Or, the sequence (AA¹)_{c} is designed for selective cleavage by urokinase or tryptase.

As one illustrative example, CD10, also known as neprilysin, neutral endopeptidase (NEP), and common acute lymphoblastic leukemia antigen (CALLA), is a type II cell-surface zinc-dependent metalloprotease. Cleavable substrates suitable for use with CD10 include Leu-Ala-Leu and Ile-Ala-Leu.

Another illustrative example is based on matrix metalloproteases (MMP). Probably the best characterized proteolytic enzymes associated with tumors, there is a clear correlation of activation of MMPs within tumor microenvironments. In particular, the soluble matrix enzymes MMP2 (gelatinase A) and MMP9 (gelatinase B), have been intensively studied, and shown to be selectively activated during tissue remodeling including tumor growth. Peptide sequences designed to be cleaved by MMP2 and MMP9 have been designed and tested for conjugates of dextran and methotrexate (Chau et al., Bioconjugate Chem. 15:931-941 (2004)); PEG (polyethylene glycol) and doxorubicin (Bae et al., Drugs Exp. Clin. Res. 29:15-23 (2004)); and albumin and doxorubicin (Kratz et al., Bioorg. Med. Chem. Lett. 11:2001-2006 (2001)). Examples of suitable sequences for use with MMPs include, but are not limited to, Pro-Val-Gly-Leu-Ile-Gly (SEQ. ID NO: 21), Gly-Pro-Leu-Gly-Val (SEQ. ID NO: 22), Gly-Pro-Leu-Gly-Ile-Ala-Gly-Gln (SEQ. ID NO: 23), Pro-Leu-Gly-Leu (SEQ. ID NO: 24), Gly-Pro-Leu-Gly-Met-Leu-Ser-Gln (SEQ. ID NO: 25), and Gly-Pro-Leu-Gly-Leu-Trp-Ala-Gln (SEQ. ID NO: 26). (See, e.g., the previously cited references as well as Kline et al., Mol. Pharmaceut. 1:9-22 (2004) and Liu et al., Cancer Res. 60:6061-6067 (2000).)

Yet another example is type II transmembrane serine proteases. This group of enzymes includes, for example, hepsin, testisin, and TMPRSS4. Gln-Ala-Arg is one substrate sequence that is useful with matriptase/MT-SP1 (which is over-expressed in breast and ovarian cancers) and Leu-Ser-Arg is useful with hepsin (over-expressed in prostate and some other tumor types). (See, e.g., Lee et. al., J. Biol. Chem. 275:36720-36725 and Kurachi and Yamamoto, Handbook of Proeolytic Enzymes Vol. 2, 2^{nd} edition (Barrett A J, Rawlings N D & Woessner J F, eds) pp. 1699-1702 (2004).)

Suitable, but non-limiting, examples of peptide sequences suitable for use in the conjugates of the invention include Val-Cit, Cit-Cit, Val-Lys, Phe-Lys, Lys-Lys, Ala-Lys, Phe-Cit, Leu-Cit, Ile-Cit, Trp, Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu (SEQ ID NO: 27), Gly-Phe-Leu-Gly (SEQ. ID NO: 28), Val-Ala, Leu-Leu-Gly-Leu (SEQ ID NO: 29), Leu-Asn-Ala, and Lys-Leu-Val. Preferred peptides sequences are Val-Cit and Val-Lys.

In another embodiment, the amino acid located the closest to the drug moiety is selected from the group consisting of: Ala, Asn, Asp, Cit, Cys, Gln, Glu, Gly, Ile, Leu, Lys, Met, Phe, Pro, Ser, Thr, Trp, Tyr, and Val. In yet another embodiment, the amino acid located the closest to the drug moiety is selected from the group consisting of: Ala, Asn, Asp, Cys, Gln, Glu, Gly, Ile, Leu, Met, Phe, Pro, Ser, Thr, Trp, Tyr, and Val.

One of skill in the art can readily evaluate an array of peptide sequences to determine their utility in the present invention without resort to undue experimentation. See, for example, Zimmerman, M., et al., (1977) Analytical Biochemistry 78:47-51; Lee, D., et al., (1999) Bioorganic and Medicinal Chemistry Letters 9:1667-72; and Rano, T. A., et al., (1997) Chemistry and Biology 4:149-55.

A conjugate of this invention may optionally contain two or more linkers. These linkers may be the same or different. For example, a peptidyl linker may be used to connect the drug to the ligand and a second peptidyl linker may attach a diagnostic agent to the complex. Other uses for additional linkers include linking analytical agents, biomolecules, targeting agents, and detectable labels to the antibody-partner complex.

### Hydrazine Linkers (H)

In another embodiment, the conjugate of the invention comprises a hydrazine self-immolative linker, wherein the conjugate has the structure:

X⁴-(L⁴)ₚ-H-(L¹)ₘ-D

wherein D, L¹, L⁴, p, m, and X⁴ are as defined above and described further herein, and H is a linker comprising the structure:

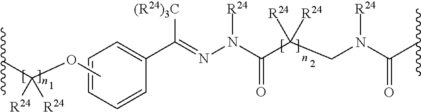

wherein n₁ is an integer from 1-10; n₂ is 0, 1, or 2; each R²⁴ is a member independently selected from the group consisting of H, substituted alkyl, unsubstituted alkyl, substituted heteroalkyl, and unsubstituted heteroalkyl; and I is either a bond (i.e., the bond between the carbon of the backbone and the adjacent nitrogen) or:

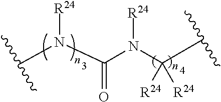

wherein n₃ is 0 or 1, with the proviso that when n₃ is 0, n₂ is not 0; and n₄ is 1, 2, or 3.

In one embodiment, the substitution on the phenyl ring is a para substitution. In preferred embodiments, nᵢ is 2, 3, or 4 or nᵢ is 3. In preferred embodiments, n₂ is 1. In preferred embodiments, I is a bond (i.e., the bond between the carbon of the backbone and the adjacent nitrogen). In one aspect, the hydrazine linker, H, can form a 6-membered self immolative linker upon cleavage, for example, when n₃ is 0 and n₄ is 2. In another aspect, the hydrazine linker, H, can form two 5-membered self immolative linkers upon cleavage. In yet other aspects, H forms a 5-membered self immolative linker, H forms a 7-membered self immolative linker, or H forms a 5-membered self immolative linker and a 6-membered self immolative linker, upon cleavage. The rate of cleavage is affected by the size of the ring formed upon cleavage. Thus, depending upon the rate of cleavage desired, an appropriate size ring to be formed upon cleavage can be selected.

Another hydrazine structure, H, has the formula:

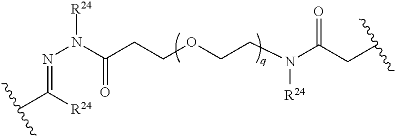

where q is 0, 1, 2, 3, 4, 5, or 6; and each R²⁴ is a member independently selected from the group consisting of H, substituted alkyl, unsubstituted alkyl, substituted heteroalkyl, and unsubstituted heteroalkyl. This hydrazine structure can also form five-, six-, or seven-membered rings and additional components can be added to form multiple rings.

The preparation, cleavage chemistry and cyclization kinetics of the various hydrazine linkers is disclosed in WO 2005/112919, the disclosure of which is incorporated herein by reference.

### Disulfide Linkers (J)

In yet another embodiment, the linker comprises an enzymatically cleavable disulfide group. In one embodiment, the invention provides a cytotoxic antibody-partner compound having a structure according to Formula (d):

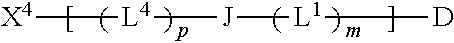

wherein D, L¹, L⁴, p, m, and X⁴ are as defined above and described further herein, and J is a disulfide linker comprising a group having the structure:

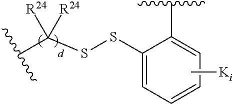

wherein each R²⁴ is a member independently selected from the group consisting of H, substituted alkyl, unsubstituted alkyl, substituted heteroalkyl, and unsubstituted heteroalkyl; each K is a member independently selected from the group consisting of substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl, unsubstituted heterocycloalkyl, halogen, NO₂, NR²¹R²², NR²¹COR²², OCONR²¹R²², OCOR²¹, and OR²¹ wherein R²¹ and R²² are independently selected from the group consisting of H, substituted alkyl, unsubstituted alkyl, substituted heteroalkyl, unsubstituted heteroalkyl, substituted aryl, unsubstituted aryl, substituted heteroaryl, unsubstituted heteroaryl, substituted heterocycloalkyl and unsubstituted heterocycloalkyl; i is an integer of 0, 1, 2, 3, or 4; and d is an integer of 0, 1, 2, 3, 4, 5, or 6.

The aromatic ring of a disulfide linker can be substituted with one or more “K” groups. A “K” group is a substituent that replaces a hydrogen otherwise attached to one of the four non-substituted carbons that are part of the ring structure. The “K” group may be a single atom, such as a halogen, or may be a multi-atom group, such as alkyl, heteroalkyl, amino, nitro, hydroxy, alkoxy, haloalkyl, and cyano. Exemplary K substituents include, but are not limited to, F, Cl, Br, I, NO₂, OH, OCH₃, NHCOCH₃, N(CH₃)₂, NHCOCF₃ and methyl. For “Kᵢ”, i is an integer of 0, 1, 2, 3, or 4. In a specific embodiment, i is 0.

In a preferred embodiment, the linker comprises an enzymatically cleavable disulfide group of the following formula:

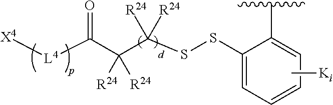

wherein L⁴, X⁴, p, and R²⁴ are as described above, and d is 0, 1, 2, 3, 4, 5, or 6. In a particular embodiment, d is 1 or 2.

A more specific disulfide linker is shown in the formula below:

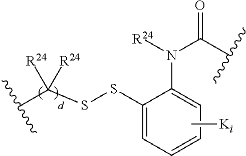

Preferably, d is 1 or 2 and each K is H.

Another disulfide linker is shown in the formula below:

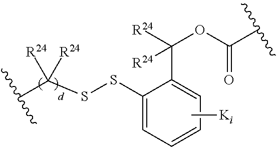

Preferably, d is 1 or 2 and each K is H.

In various embodiments, the disulfides are ortho to the amine. In another specific embodiment, a is 0. In preferred embodiments, R²⁴ is independently selected from H and CH₃.

The preparation and use of disulfide linkers such as those described above is disclosed in WO 2005/112919, the disclosure of which is incorporated herein by reference.

For further discussion of types of cytotoxins, linkers and the conjugation of therapeutic agents to antibodies, see also U.S. Pat. No. 7,087,600; U.S. Pat. No. 6,989,452; U.S. Pat. No. 7,129,261; US 2006/0004081; US 2006/0247295; WO 02/096910; WO 2007/051081; WO 2005/112919; WO 2007/059404; WO 2008/083312; WO 2008/103693; Saito et al. (2003) Adv. Drug Deliv. Rev. 55:199-215; Trail et al. (2003) Cancer Immunol. Immunother. 52:328-337; Payne. (2003) Cancer Cell 3:207-212; Allen (2002) Nat. Rev. Cancer 2:750-763; Pastan and Kreitman (2002) Curr. Opin. Investig. Drugs 3:1089-1091; Senter and Springer (2001) Adv. Drug Deliv. Rev. 53:247-264, each of which is hereby incorporated by reference.

### Cytotoxins as Partner Molecules

In one aspect, the present invention features an antibody conjugated to a partner molecule, such as a cytotoxin, a drug (e.g., an immunosuppressant) or a radiotoxin. Such conjugates are also referred to as “immunotoxins.” A cytotoxin or cytotoxic agent includes any agent that is detrimental to (e.g., kills) cells. Herein, “cytotoxin” includes compounds that are in a prodrug form and are converted in vivo to the actual toxic species.

Examples of partner molecules of the present invention include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Examples of partner molecules also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, tubulysin, dibromomannitol, streptozotocin, mitomycin C, cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other preferred examples of partner molecules that can be conjugated to an antibody of the invention include calicheamicins, maytansines and auristatins, and derivatives thereof.

Preferred examples of partner molecule are analogs and derivatives of CC-1065 and the structurally related duocarmycins. Despite its potent and broad antitumor activity, CC-1065 cannot be used in humans because it causes delayed death in experimental animals, prompting a search for analogs or derivatives with a better therapeutic index.

Many analogues and derivatives of CC-1065 and the duocarmycins are known in the art. The research into the structure, synthesis and properties of many of the compounds has been reviewed. See, for example, Boger et al., Angew. Chem. Int. Ed. Engl. 35: 1438 (1996); and Boger et al., Chem. Rev. 97: 787 (1997). Other disclosures relating to CC-1065 analogs or derivatives include: U.S. Pat. No. 5,101,038; U.S. Pat. No. 5,641,780; U.S. Pat. No. 5,187,186; U.S. Pat. No. 5,070,092; U.S. Pat. No. 5,703,080; U.S. Pat. No. 5,070,092; U.S. Pat. No. 5,641,780; U.S. Pat. No. 5,101,038; U.S. Pat. No. 5,084,468; U.S. Pat. No. 5,739,350; U.S. Pat. No. 4,978,757, U.S. Pat. No. 5,332,837 and U.S. Pat. No. 4,912,227; WO 96/10405; and EP 0,537,575 A1

In a particularly preferred aspect, the partner molecule is a CC-1065/duocarmycin analog having a structure according to the following formula (e):

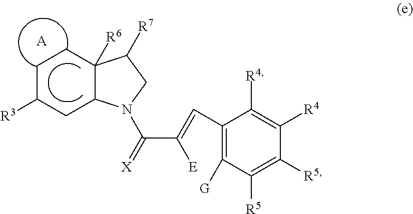

in which ring system A is a member selected from substituted or unsubstituted aryl substituted or unsubstituted heteroaryl and substituted or unsubstituted heterocycloalkyl groups. Exemplary ring systems A include phenyl and pyrrole.

The symbols E and G are independently selected from H, substituted or unsubstituted alkyl, substituted or unsubstituted heteroalkyl, a heteroatom, a single bond or E and G are optionally joined to form a ring system selected from substituted or unsubstituted aryl, substituted or unsubstituted heteroaryl and substituted or unsubstituted heterocycloalkyl.

The symbol X represents a member selected from O, S and NR²³. R²³ is a member selected from H, substituted or unsubstituted alkyl, substituted or unsubstituted heteroalkyl, and acyl.

The symbol R³ represents a member selected from (═O), Se, NHR¹¹ and OR¹¹, in which R¹¹ is H, substituted or unsubstituted alkyl, substituted or unsubstituted heteroalkyl, monophosphates, diphosphates, triphosphates, sulfonates, acyl, C(O)R¹²R¹³, C(O)OR¹², C(O)NR¹²R¹³, P(O)(OR¹²)₂, C(O)CHR¹²R¹³, SR¹² or SiR¹²R¹³R¹⁴. The symbols R¹², R¹³, and R¹⁴ independently represent H, substituted or unsubstituted alkyl, substituted or unsubstituted heteroalkyl and substituted or unsubstituted aryl, where R¹² and R¹³ together with the nitrogen or carbon atom to which they are attached are optionally joined to form a substituted or unsubstituted heterocycloalkyl ring system having from 4 to 6 members, optionally containing two or more heteroatoms.

R⁴, R^{4′}, R⁵ and R^{5′} are members independently selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20, or any adjacent pair of R⁴, R^{4′}, R⁵ and R^{5′}, together with the carbon atoms to which they are attached, are joined to form a substituted or unsubstituted cycloalkyl or heterocycloalkyl ring system having from 4 to 6 members. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms. One exemplary structure is aniline.

One of R³, R⁴, R^{4′}, R⁵, and R^{5′} joins the cytotoxin to a linker or enzyme cleavable substrate of the present invention, as described herein, for example to L¹ or L³, if present or to F, H, or J.

R⁶ is a single bond which is either present or absent. When R⁶ is present, R⁶ and R⁷ are joined to form a cyclopropyl ring. R⁷ is CH₂—X¹ or —CH₂—. When R⁷ is —CH₂— it is a component of the cyclopropane ring. The symbol X′ represents a leaving group such as a halogen, for example Cl, Br or F. The combinations of R⁶ and R⁷ are interpreted in a manner that does not violate the principles of chemical valence.

X¹ may be any leaving group. Useful leaving groups include, but are not limited to, halogens, azides, sulfonic esters (e.g., alkylsulfonyl, arylsulfonyl), oxonium ions, alkyl perchlorates, ammonioalkanesulfonate esters, alkylfluorosulfonates and fluorinated compounds (e.g., triflates, nonaflates, tresylates) and the like. Particular halogens useful as leaving groups are F, Cl and Br.

The curved line within the six-membered ring indicates that the ring may have one or more degrees of unsaturation, and it may be aromatic. Thus, ring structures such as those set forth below, and related structures, are within the scope of Formula (f):

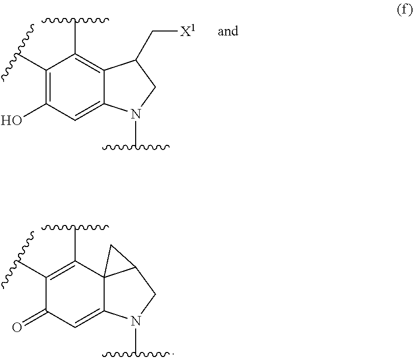

In one embodiment, R¹¹ includes a moiety, X⁵, that does not self-cyclize and links the drug to L¹ or L³, if present, or to F, H, or J. The moiety, X⁵, is preferably cleavable using an enzyme and, when cleaved, provides the active drug. As an example, R¹¹ can have the following structure (with the right side coupling to the remainder of the drug):

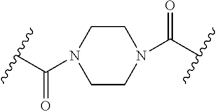

In some embodiments, at least one of R⁴, R^{4′}, R⁵, and R^{5′} links said drug to L¹, if present, or to F, H, J, or X², and R³ is selected from SR″, NHR¹¹ and OR″.R¹¹ is selected from —SO(OH)₂, —PO(OH)₂, —Si(CH₃)₂C(CH₃)₃, —C(O)OPhNH(AA)ₘ,

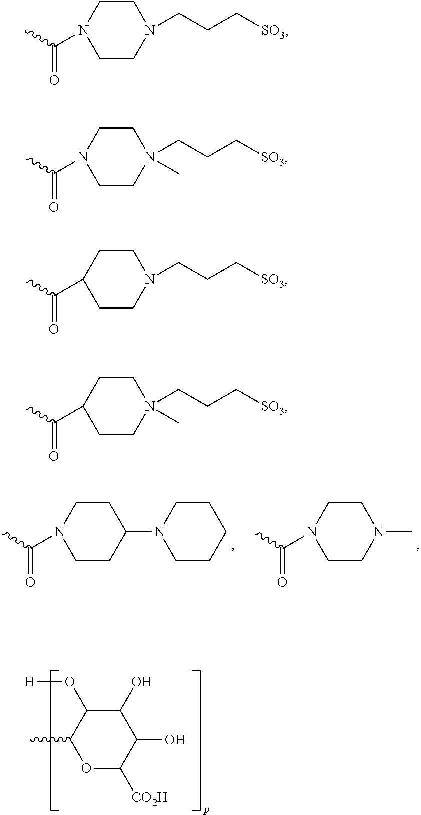

or any other sugar or combination of sugars

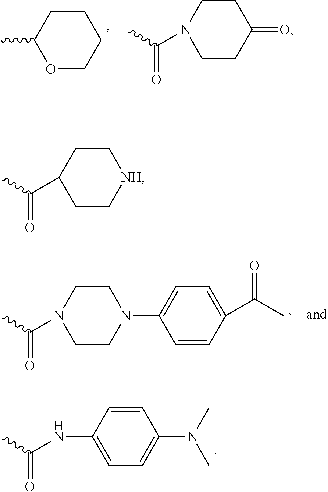

and pharmaceutically acceptable salts thereof, where n is any integer in the range of 1 to 10, m is any integer in the range of 1 to 4, p is any integer in the range of 1 to 6, and AA is any natural or non-natural amino acid. Where the compound of formula (e) is conjugated via R⁴, R^{4′}, R⁵, or R⁶, R³ preferably comprises a cleavable blocking group whose presence blocks the cytotoxic activity of the compound but is cleavable under conditions found at the intended site of action by a mechanism different from that for cleavage of the linker conjugating the cytotoxin to the antibody. In this way, if there is adventitiouis cleavage of the conjugate in the plasma, the blocking group attenuates the cytotoxicity of the released cytotoxin. For instance, if the conjugate has a hydrazone or disulfide linker, the blocking group can be an enzymatically cleavable amide. Or, if the linker is a peptidyl one cleavable by a protease, the blocking group can be an ester or carbamate cleavable by a carboxyesterase.

For example, in a preferred embodiment, D is a cytotoxin having a structure (j):

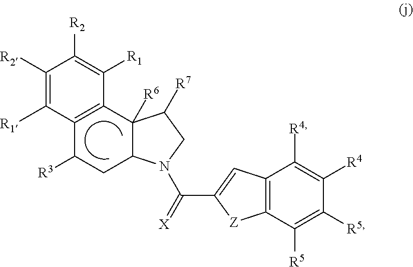

In this structure, R³, R⁶, R⁷, R⁴, R^{4′}, R⁵, R^{5′} and X are as described above for Formula (e). Z is a member selected from O, S and NR²³, where R²³ is a member selected from H, substituted or unsubstituted alkyl, substituted or unsubstituted heteroalkyl, and acyl.

R¹ is H, substituted or unsubstituted lower alkyl, C(O)R⁸, or CO₂R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R^{1′} is H, substituted or unsubstituted lower alkyl, or C(O)R⁸, wherein R⁸ is a member selected from NR⁹R¹⁰ and OR⁹, in which R⁹ and R¹⁰ are members independently selected from H, substituted or unsubstituted alkyl and substituted or unsubstituted heteroalkyl.

R² is H, or substituted or unsubstituted lower alkyl or unsubstituted heteroalkyl or cyano or alkoxy; and R^{2′} is H, or substituted or unsubstituted lower alkyl or unsubstituted heteroalkyl.

One of R³, R⁴, R^{4′}, R⁵, or R^{5′} links the cytotoxin to L¹ or L³, if present, or to F, H, or J.

A further embodiment has the formula:

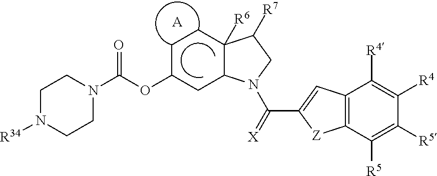

In this structure, A, R⁶, R⁷, X, R⁴, R^{4′}, R⁵, and R^{5′} are as described above for Formula (e). Z is a member selected from O, S and NR²³, where R²³ is a member selected from H, substituted or unsubstituted alkyl, substituted or unsubstituted heteroalkyl, and acyl;

R³⁴ is C(═O)R³³ or C₁-C₆ alkyl, where R³³ is selected from H, substituted or unsubstituted alkyl, substituted or unsubstituted aryl, substituted or unsubstituted heteroaryl, substituted or unsubstituted heterocycloalkyl, halogen, NO₂, NR¹⁵R¹⁶, NC(O)R¹⁵, OC(O)NR¹⁵R¹⁶, OC(O)OR¹⁵, C(O)R¹⁵, SR¹⁵, OR¹⁵, CR¹⁵═NR¹⁶, and O(CH₂)ₙN(CH₃)₂, where n is an integer from 1 to 20. R¹⁵ and R¹⁶ independently represent H, substituted or unsubstituted alkyl, substituted or unsubstituted heteroalkyl, substituted or unsubstituted aryl, substituted or unsubstituted heteroaryl, substituted or unsubstituted heterocycloalkyl and substituted or unsubstituted peptidyl, where R¹⁵ and R¹⁶ together with the nitrogen atom to which they are attached are optionally joined to form a substituted or unsubstituted heterocycloalkyl ring system having from 4 to 6 members, optionally containing two or more heteroatoms.

Preferably, A is substituted or unsubstituted phenyl or substituted or unsubstituted pyrrole. Further, any selection of substituents described herein for R¹¹ is also applicable to R³³.

A preferred partner molecule has a structure represented by formula (I)

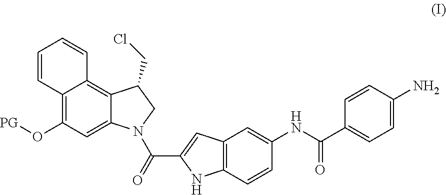

In formula (I), PD represents a prodrugging group (sometimes also referred to as a protecting group). Compound (I) is hydrolyzed in situ (preferably enzymatically) to release the compound of formula (II). As those skilled in the art will recognize, compound (II) belongs to the class of compounds known as CBI compounds (Boger et al., J. Org. Chem. 2001, 66, 6654-6661 and Boger et al., US 2005/0014700 A1 (2005). CBI compounds are converted in situ (or, when administered to a patient, in vivo) to their cyclopropyl derivatives such as compound (III), bind to the minor groove of DNA, and then alkylate DNA on an adenine group, with the cyclopropyl derivative believed to be the actual alkylating species.

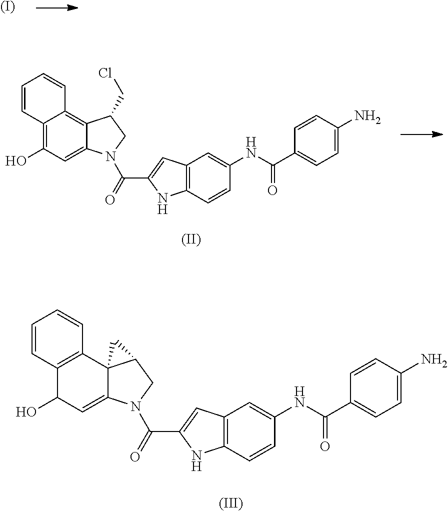

Non-limiting examples of suitable prodrugging groups PD include esters, carbamates, phosphates, and glycosides, as illustrated following:

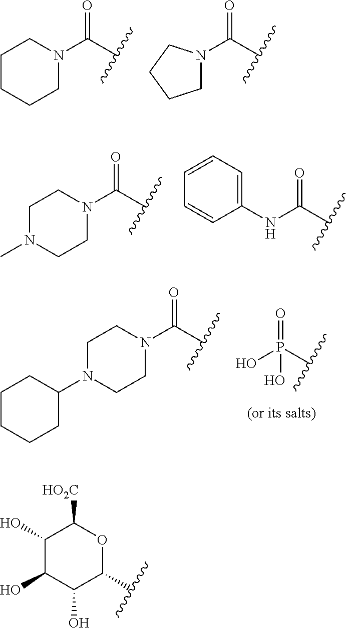

Preferred prodrugging groups PD are carbamates (exemplified by the first five structures above), which are hydrolyzable by carboxyesterases; phosphates (the sixth structure above), which are hydrolyzable by alkaline phosphatase, and β-glucuronic acid derivatives, which are hydrolyzable by β-glucuronidase. A specific preferred partner molecule is a carbamate prodrugged one, represented by formula (IV):

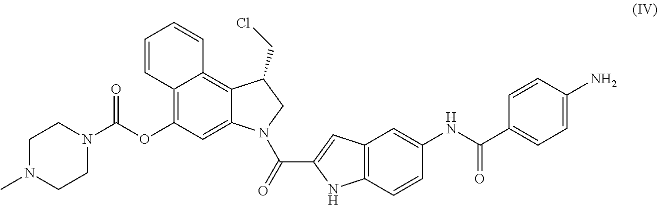

### Markers as Partner Molecules

Where the partner molecule is a marker, it can be any moiety having or generating a detectable physical or chemical property indicating its presence in a particular tissue or cell. Markers (sometimes also called reporter groups) have been well developed in the area of immunoassays, biomedical research, and medical diagnosis. A marker may be detected by spectroscopic, photochemical, biochemical, immunochemical, electrical, optical or chemical means. Examples include magnetic beads (e.g., DYNABEADS®), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The marker is preferably a member selected from the group consisting of radioactive isotopes, fluorescent agents, fluorescent agent precursors, chromophores, enzymes and combinations thereof. Examples of suitable enzymes are horseradish peroxidase, alkaline phosphatase, β-galactosidase, and glucose oxidase. Fluorescent agents include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Markers can be attached by indirect means: a ligand molecule (e.g., biotin) is covalently bound to an antibody. The ligand then binds to another molecule (e.g., streptavidin), which is either inherently detectable or covalently bound to a signal system, such as a detectable enzyme, a fluorescent compound, or a chemiluminescent compound.

### Examples of Conjugates

Specific examples of partner molecule-linker combinations suitable for conjugation to an antibody of this invention are shown following:

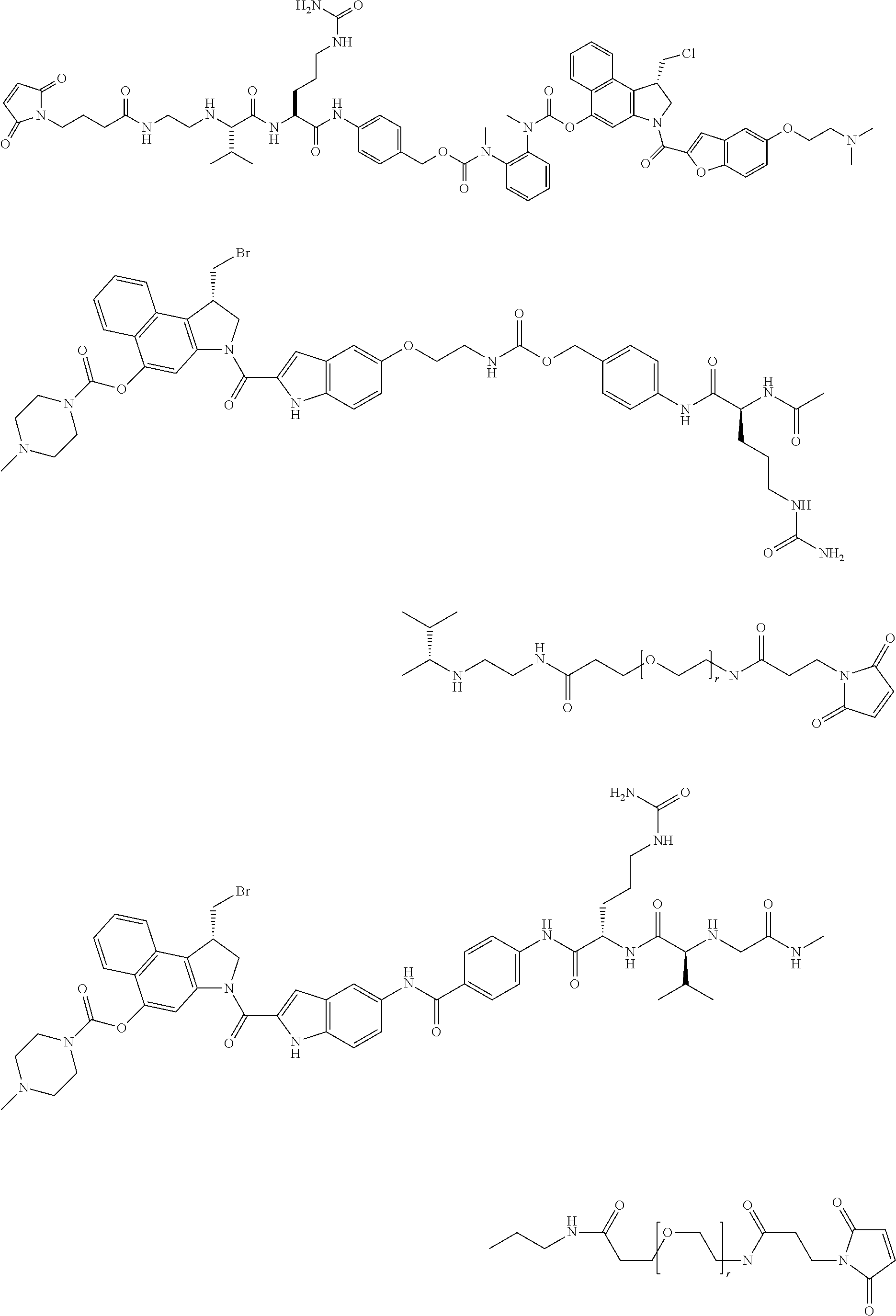

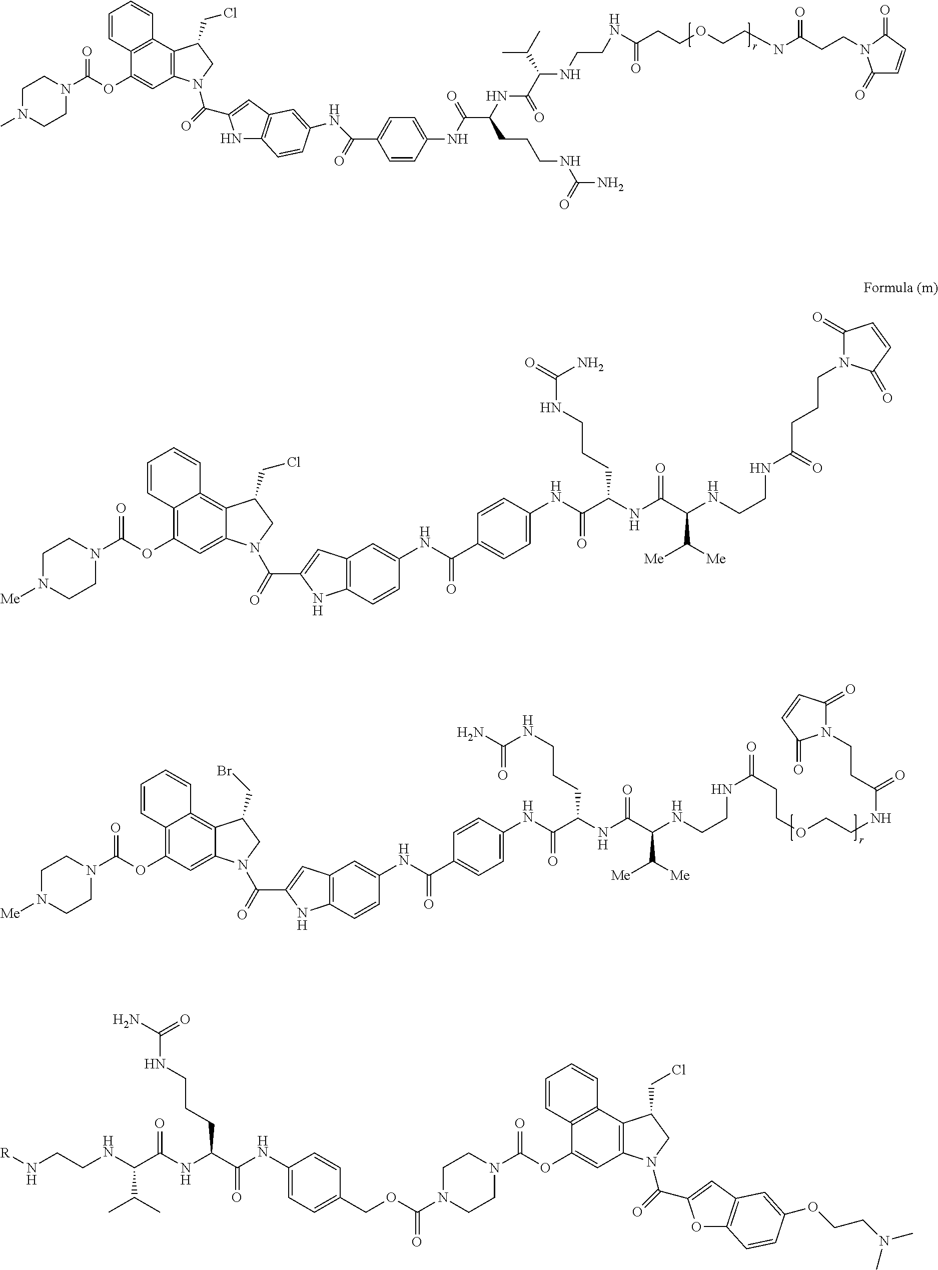

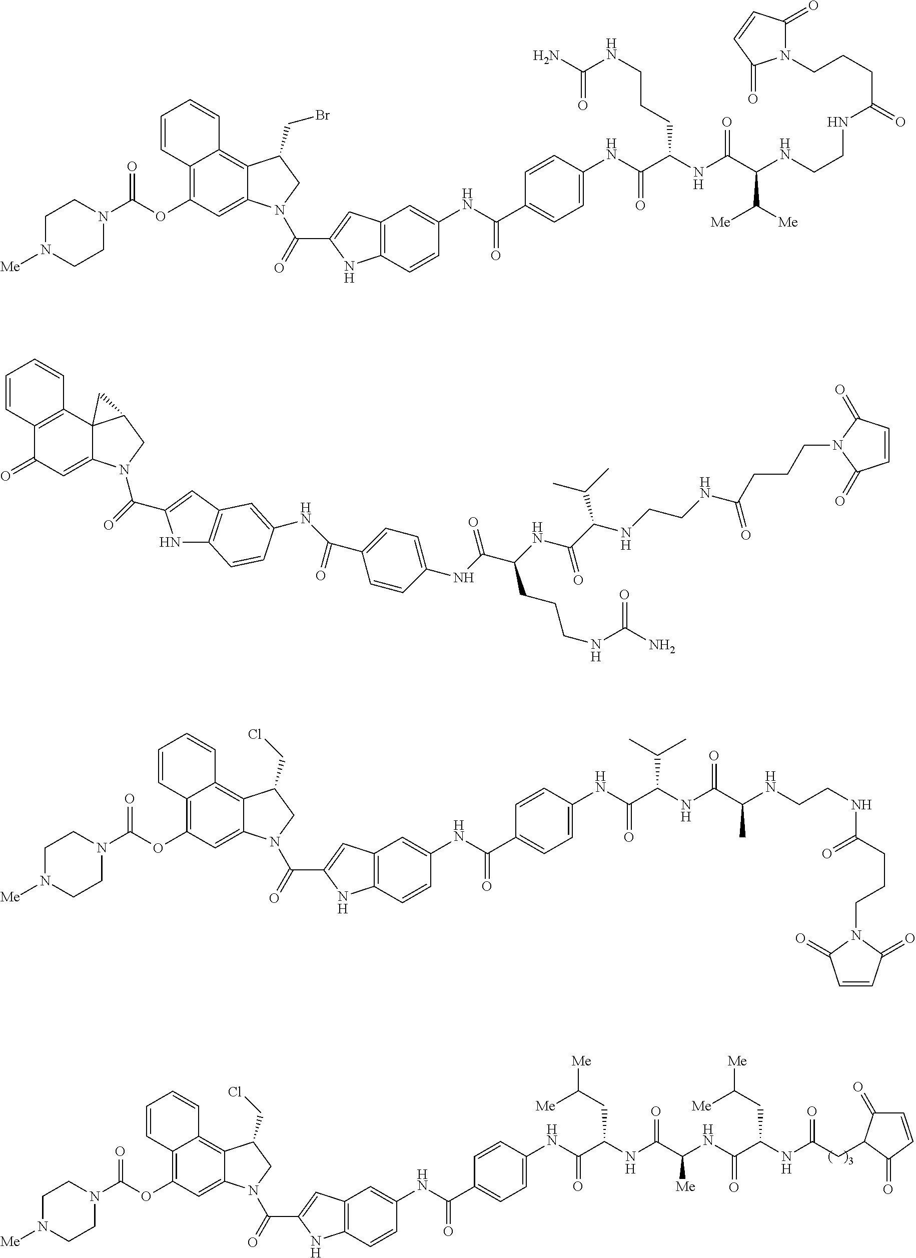

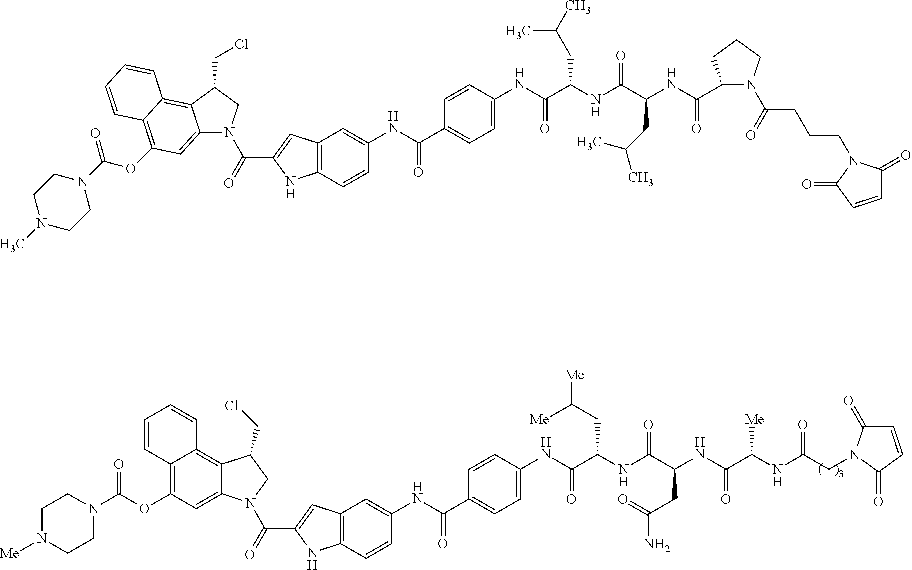

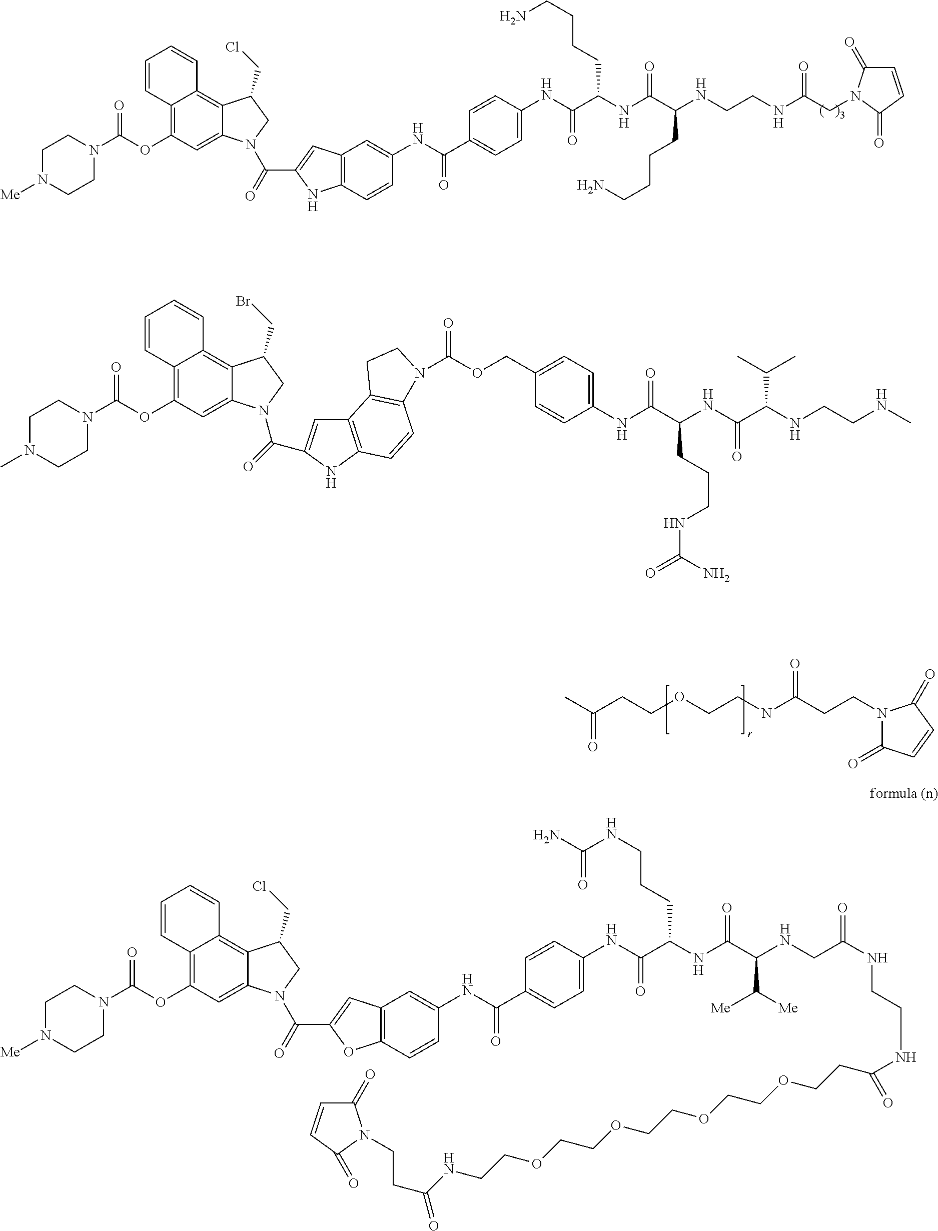

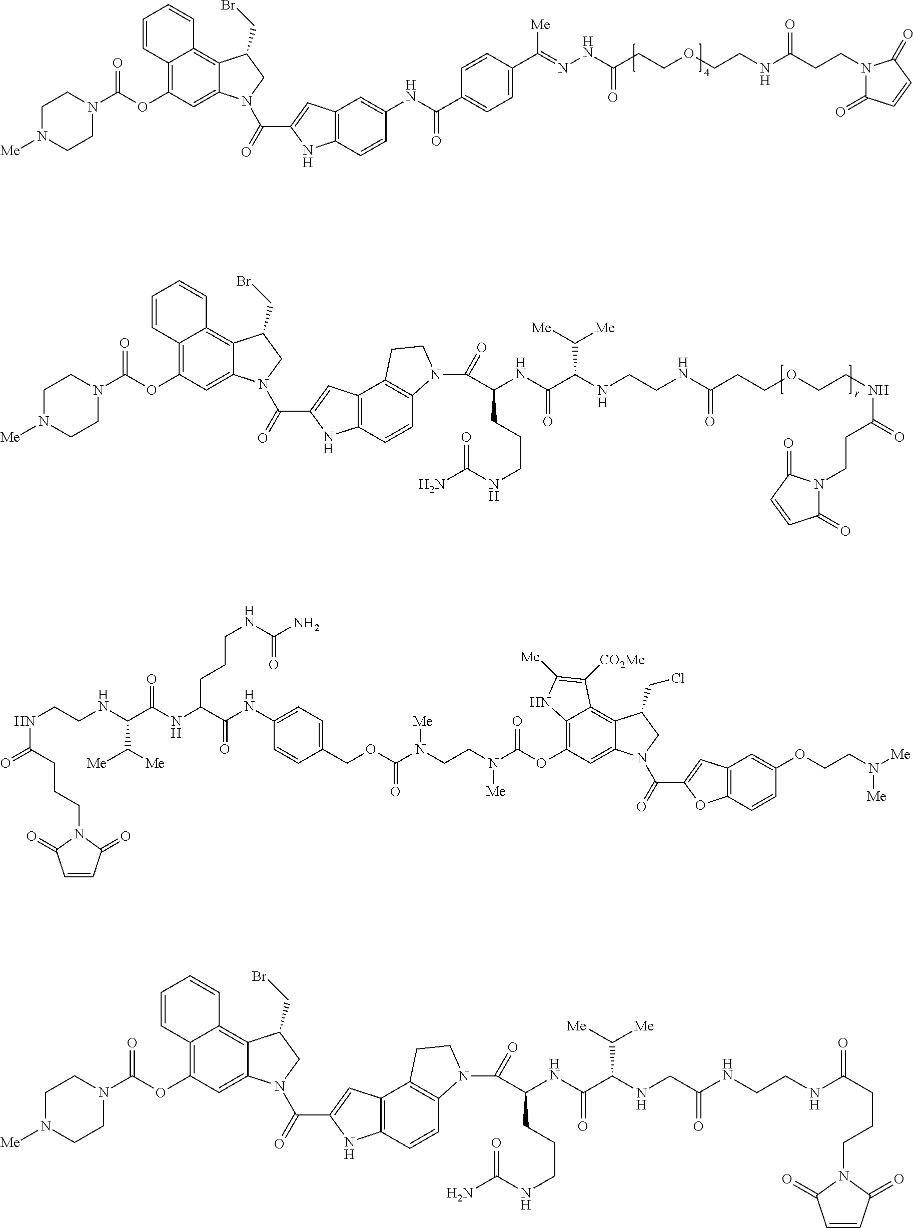

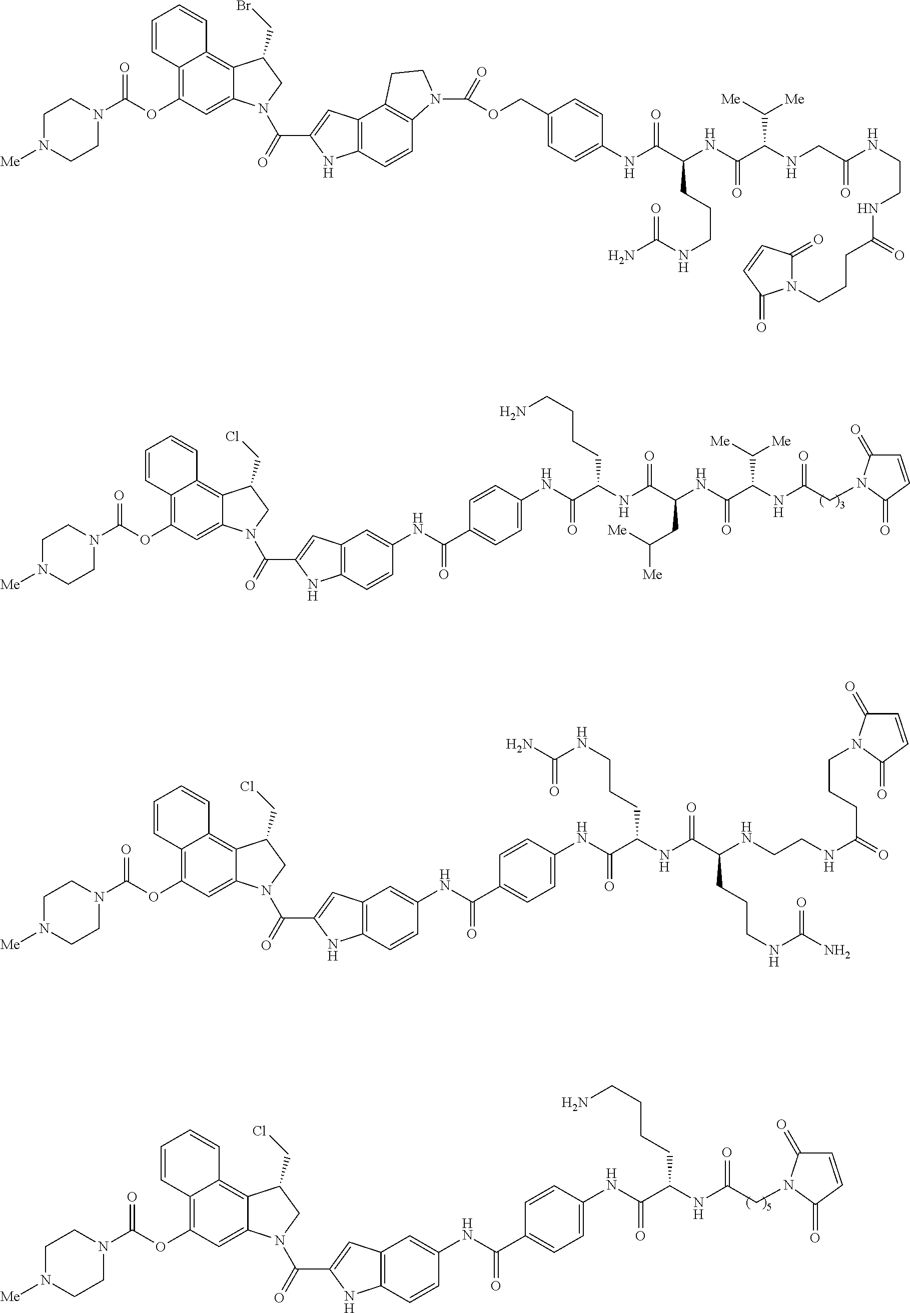

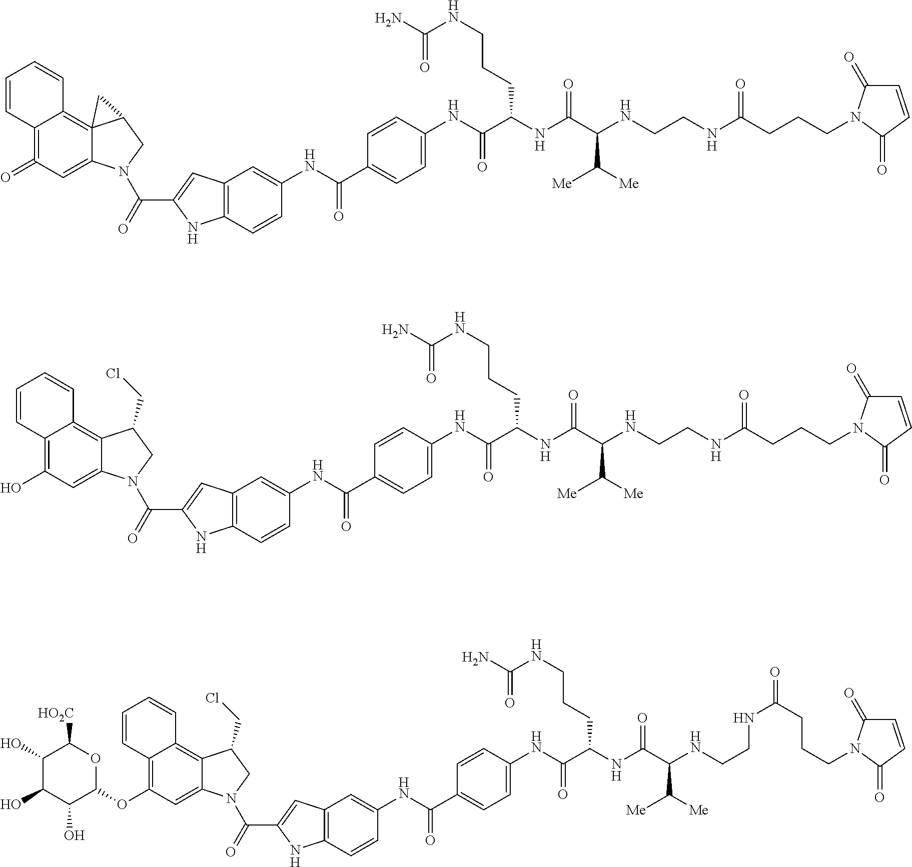

In the foregoing compounds, where the subscript r is present in a formula, it is an integer in the range of 0 to 24. R, wherever it occurs, is

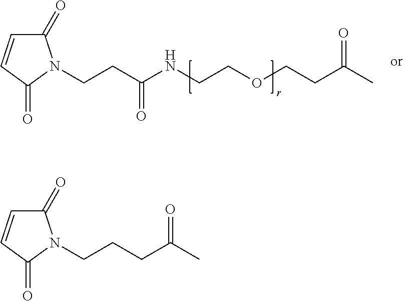

Each of the foregoing compounds has a maleimide group and is ready for conjugation to an antibody via a suithydryl group thereon.

### Pharmaceutical Compositions

In another aspect, the present invention provides a pharmaceutical composition containing a conjugate of the present invention formulated together with a pharmaceutically acceptable carrier and, optionally, other active or inactive ingredients.

Pharmaceutical compositions of the invention also can be administered in combination therapy with other agents. For example, the combination therapy can include an anti-RG-1 conjugate of the present invention combined with at least one other anti-inflammatory or immunosuppressant agent. Examples of therapeutic agents that can be used in combination therapy are described in greater detail below.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable carriers include water, ethanol, polyols (such as glycerol, propylene glycol, polyethylene glycol, and the like), and mixtures thereof, vegetable oils such as olive oil, and injectable organic esters, such as ethyl oleate. Proper fluidity can be maintained by the use of coating materials, such as lecithin, by the maintenance of the required particle size in the case of dispersions, and by the use of surfactants.

The compositions may also contain adjuvants such as preservatives, wetting agents, emulsifying agents and dispersing agents. Prevention of presence of microorganisms may be ensured both by sterilization and by the inclusion of antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, e.g., water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient that can be combined with a carrier to produce a single dosage form will vary depending upon the subject being treated and the particular mode of administration and will generally be that amount of the composition that produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of a conjugate, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for conjugate of the invention include 1 mg/kg body weight or 3 mg/kg body weight via intravenous administration, with the conjugate being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma conjugate concentration of about 1-1000 μg/ml and in some methods about 25-300 μg/ml.

Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime

For use in the prophylaxis and/or treatment of diseases related to abnormal cellular proliferation, a circulating concentration of administered compound of about 0.001 μM to 20 μM is preferred, with about 0.01 μM to 5 μM being preferred.

Patient doses for oral administration of the compounds described herein, typically range from about 1 mg/day to about 10,000 mg/day, more typically from about 10 mg/day to about 1,000 mg/day, and most typically from about 50 mg/day to about 500 mg/day. Stated in terms of patient body weight, typical dosages range from about 0.01 to about 150 mg/kg/day, more typically from about 0.1 to about 15 mg/kg/day, and most typically from about 1 to about 10 mg/kg/day, for example 5 mg/kg/day or 3 mg/kg/day.

In some embodiments, patient doses that retard or inhibit tumor growth can be 1 mmol/kg/day or less. For example, the patient doses can be 0.9, 0.6, 0.5, 0.45, 0.3, 0.2, 0.15, or 0.1 mmol/kg/day or less (referring to moles of the drug). Preferably, the antibody-drug conjugate retards growth of the tumor when administered in the daily dosage amount over a period of at least five days. In at least some embodiments, the tumor is a human-type tumor in a SCID mouse. As an example, the SCID mouse can be a CB17.SCID mouse (available from Taconic, Germantown, N.Y.).

Actual dosage levels may be varied so as to obtain an amount of the active ingredient effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion, the duration of the treatment, other drugs, compounds and/or materials used in combination with the particular compositions employed, the age, sex, weight, condition, general health and prior medical history of the patient, and like factors.

A “therapeutically effective dosage” of a conjugate of the invention preferably results in a decrease in severity of disease symptoms, an increase in frequency and duration of disease symptom-free periods, and/or a prevention of impairment or disability due to the disease affliction. For example, for the treatment of RG-1⁺ tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a conjugate to inhibit tumor growth can be evaluated in an animal model system predictive of efficacy in human tumors. Alternatively, this property of a composition can be evaluated by examining its ability to inhibit cell growth, such ability being measurable in vitro by assays known to the skilled practitioner. A therapeutically effective amount of a therapeutic compound can decrease tumor size, or otherwise ameliorate symptoms in a subject. One of ordinary skill in the art can determine such amounts based on such factors as the subject's size, the severity of symptoms, and the particular composition or route of administration selected.

A conjugate of this invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically.

The active compounds can be prepared with carriers that will protect them against premature release, such as a controlled release formulation, implants, transdermal patches, and micro encapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as disclosed in U.S. Pat. Nos. 5,399,163; 5,383,851; 5,312,335; 5,064,413; 4,941,880; 4,790,824; or 4,596,556. Examples of other suitable devices include those disclosed in: U.S. Pat. No. 4,487,603; U.S. Pat. No. 4,486,194; U.S. Pat. No. 4,447,233; U.S. Pat. No. 4,447,224; U.S. Pat. No. 4,439,196; and U.S. Pat. No. 4,475,196. These patents are incorporated herein by reference.

In certain embodiments, the conjugates of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### Uses and Methods

The antibody-partner molecule conjugate compositions and methods of the present invention have numerous in vitro and in vivo diagnostic and therapeutic utilities involving the diagnosis and treatment of RG-1 mediated disorders. For example, these molecules can be administered to cells in culture, in vitro or ex vivo, or to human subjects, e.g., in vivo, to treat, prevent and to diagnose a variety of disorders. Preferred subjects include human patients having disorders mediated by RG-1 activity. The methods are particularly suitable for treating human patients having a disorder associated with aberrant RG-1 expression. Noteworthy among these are prostate and bladder cancers. We have also found that RG-1 is associated with a number of other tumor types, notably lung cancer, gastric cancer, breast cancer, renal cancer, pancreatic cancer, colon cancer, melanoma, and insulinoma. In most cases the RG-1 is found on the tumor stroma, although in the cases of pancreatic cancer and insulinoma there was expression on the tumor cells. Thus, the conjugates of this invention are suitable for treating such cancers.

Given the specific binding of the antibodies of the invention for RG-1, the antibodies of the invention can be used to specifically detect RG-1 expression and, moreover, can be used to purify RG-1 via immunoaffinity purification.

Furthermore, given the expression of RG-1 by various tumor cells, the antibody-partner molecule conjugate compositions and methods of the present invention can be used to treat a subject with a tumorigenic disorder, e.g., a disorder characterized by the presence of tumor cells expressing RG-1 including, for example, prostate cancer or bladder cancer.

In one embodiment, the compositions of the invention can be used to detect levels of RG-1, which levels can then be linked to certain disease symptoms. Alternatively, the compositions can be used to inhibit or block RG-1 function which, in turn, can be linked to the prevention or amelioration of certain disease symptoms, thereby implicating RG-1 as a mediator of the disease. This can be achieved by contacting a sample and a control sample with the anti-RG-1 antibody under conditions that allow for the formation of a complex between the antibody and RG-1. Any complexes formed between the antibody and RG-1 are detected and compared in the sample and the control, for example using the partner molecule as a marker or reporting group.

In another embodiment, the compositions of the invention can be initially tested for binding activity associated with therapeutic or diagnostic use in vitro. For example, compositions of the invention can be tested using flow cytometric assays known in the art.

In a particular embodiment, the compositions are used in vivo to treat, prevent or diagnose a variety of RG-1-related diseases. Examples of RG-1-related diseases include, among others, prostate cancer and bladder cancer.

As previously described, the compositions of the invention can comprise agents including, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/kg dose once every four weeks and adriamycin is intravenously administered as a 60-75 mg/ml dose once every 21 days. Co-administration of the human anti-RG-1 antibodies, or antigen binding fragments thereof, of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Where the antibody has complement binding sites, such as portions from IgG1, -2, or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The compositions of the invention can also be administered together with complement. In certain embodiments, the instant disclosure provides compositions comprising human antibodies, multispecific or bispecific molecules and serum or complement. These compositions can be advantageous when the complement is located in close proximity to the human antibodies, multispecific or bispecific molecules. Alternatively, the human antibodies, multispecific or bispecific molecules of the invention and the complement or serum can be administered separately.

Also within the scope of the present invention are kits comprising the antibody compositions of the invention and instructions for use. The kit can further contain one or more additional reagents, such as an immunosuppressive reagent, a cytotoxic agent or a radiotoxic agent, or one or more additional human antibodies of the invention (e.g., a human antibody having a complementary activity which binds to an epitope in the RG-1 antigen distinct from the first human antibody).

Accordingly, patients treated with antibody compositions of the invention can be additionally administered (prior to, simultaneously with, or following administration of a composition of the invention) with another therapeutic agent, such as a cytotoxic or radiotoxic agent, which enhances or augments the therapeutic effect of the composition.

In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

The compositions of the invention can also be used to target cells expressing FcγR or RG-1, for example for labeling such cells. For such use, the binding agent can be linked to a molecule that can be detected. Thus, the invention provides methods for localizing ex vivo or in vitro cells expressing Fc receptors, such as FcγR, or RG-1. The detectable label can be, e.g., a radioisotope, a fluorescent compound, an enzyme, or an enzyme co-factor.

In yet another embodiment, immunoconjugates of the invention can be used to target compounds (e.g., therapeutic agents, labels, cytotoxins, radiotoxoins immunosuppressants, etc.) to cells which express RG-1 by linking such compounds to the antibody. For example, an anti-RG-1 antibody can be conjugated to any of the cytotoxin compounds described in U.S. Pat. Nos. 6,281,354 and 6,548,530, US 2003/0050331, 2003/0064984, 2003/0073852, and 2004/0087497, or WO 03/022806. Thus, the invention also provides methods for localizing ex vivo or in vivo cells expressing RG-1 (e.g., with a detectable label, such as a radioisotope, a fluorescent compound, an enzyme, or an enzyme co-factor). Alternatively, the immunoconjugates can be used to kill cells which have RG-1 cell surface receptors by targeting cytotoxins or radiotoxins to RG-1.

In yet another embodiment, the immunoconjugates of the invention comprise hydrazone linkers that are readily cleaved at endosomal pHs found inside the cell. Such linkers can be useful for non-internalizing conjugates since the extracellular milieu of tumors is hypoxic and acidic. Measurement of the extracellular pH (pHe) of tumors has been carried out by a number of groups. pHe is believed to be controlled by the limited availability of oxygen and glucose in the tumor microenvironment leading to build up of lactate and CO₂ (Helmlinger et al., (2002). Clin. Cancer Res. 8, 1284-1291). Estimates of pile are consistently around 6.8; or 0.5 pH units less than the corresponding tissue (Yamagata & Tannock (1996) Br. J. Cancer 73, 1328-1334; Yamagata et al., (1998). Br. J. Cancer 77, 1726-1731; Helmlinger et al., (1997) Nature Med. 3, 177-182). On manipulation it is possible to push the pH as low as 6.5 for a short period (Helmlinger et al., (1997) Nature Med. 3, 177-182; Kozin et al., (2001) Cancer Res. 61, 4740-4743). Although this is a small differential in pH, the antibody will retain the drug in the tumor environment for an extended period which will allow release of the drug from the hydrazone linker. There is a strong correlation with pO2 and pile, with the lowest pHe values being found in necrotic poorly vascularized areas in the center of the tumor—those areas which antibodies penetrate to the least, but can be retained for long periods (Yamagata et al., (1998) Br. J. Cancer 77, 1726-1731).

The present invention is further illustrated by the following examples which should not be construed as further limiting. The contents of all figures and all references, patents and published patent applications cited throughout this application are expressly incorporated herein by reference.

### Example 1

### Preparation and Formulation of Conjugates

Anti-RG-1 antibody 19G9 and several comparative antibodies were conjugated to the molecule of formula (m):

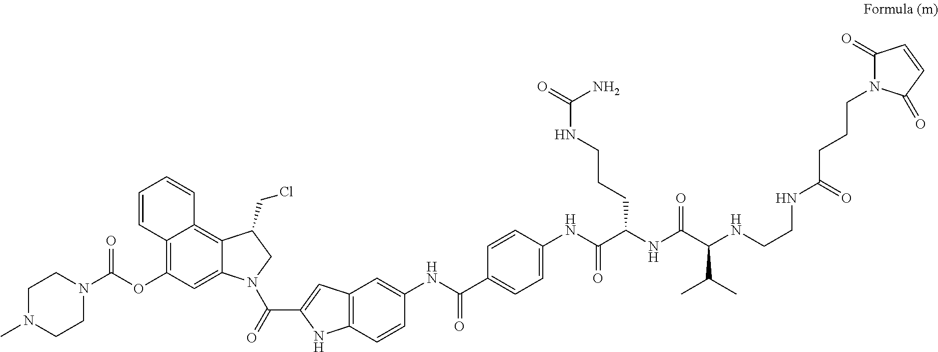

The following procedure, employed for antibody 19G9 and molecule (m), is representative.

Antibody 19G9 at a concentration of ˜5 mg/mL in 100 mM sodium phosphate, 50 mM NaCl, 2 mM DTPA, pH 8.0, is thiolated with a 10-fold molar excess of 2-iminothiolane. The thiolation reaction was allowed to proceed for 1 hour at room temperature with continuous mixing. (2-Iminothiolane reacts with lysine ε-amino groups and converts them into a thiol usable in conjugation reactions.)

Following thiolation, the antibody is buffer exchanged into conjugation buffer (50 mM HEPES, 5 mM Glycine, 2 mM DTPA, pH 5.5) by diafiltration using a 10 kDa NMWO flat sheet Tangential Flow Filtration (TFF) cassette with a PES membrane. The concentration of the thiolated antibody is adjusted to 2.5 mg/mL and thiol concentration is determined.

A 5 mM stock solution of molecule (m) in DMSO is added at a 3-fold molar excess per thiol of antibody and mixed for 90 minutes at room temperature. The conjugated antibody is filtered through a 0.2 μm filter. Following conjugation, 100 mM of N-ethylmaleimide in DMSO is added at a 10-fold molar excess of antibody thiol content to quench any unreacted thiol groups. This quenching reaction is done for one hour at room temperature with continuous mixing.

The conjugate is filtered through a 0.2 μm filter prior to cation-exchange chromatographic purification. The SP Sepharose High Performance Cation Exchange column (CEX) is regenerated with 5 CVs (column volumes) of 50 mM HEPES, 5 mM Glycine, 1M NaCl, pH 5.5. Following regeneration, the column is equilibrated with 3 CVs of equilibration buffer (50 mM HEPES, 5 mM Glycine, pH 5.5). The conjugate is loaded and the column is washed once with the equilibration buffer. The conjugate is eluted with 50 mM HEPES, 5 mM Glycine, 230 mM NaCl, pH 5.5. Eluate is collected in fractions. The column is then regenerated with 50 mM HEPES, 5 mM Glycine, 1M NaCl, pH 5.5 to remove protein aggregates and any unreacted molecule (m).

Pooling of eluate fractions is based on aggregation levels and Substitution Ratios (SR) i.e. mole of partner molecule per mole of antibody. The pooling criteria are ≧95% monomer determined by SEC-HPLC with an SR range of 1-1.5.

The purified CEX eluate pool is buffer exchanged into bulk diafiltration buffer (30 mg/mL sucrose, 10 mg/mL glycine, pH 6.0) in a 10 NMWCO flat-sheet TFF cassette with a PES membrane. Bulk formulation is completed by dilution of the protein concentration to 5 mg/ml and by the addition of Dextran 40 to the diafiltered conjugate solution to a final concentration of 10 mg/ml. The formulated bulk is filtered through a 0.2 μm PES filter into sterile PETG bottles and stored at 2 to 8° C.

The conjugate of an antibody with the molecule of formula (m) can be represented by formula (A), where Ab denotes the antibody (in the example above, the antibody being 19G9). As noted by the SR range of 1 to 1.5, some of the antibodies have more than one molecule of formula (m) attached thereto, the range of 1 to 1.5 being a statistical average.

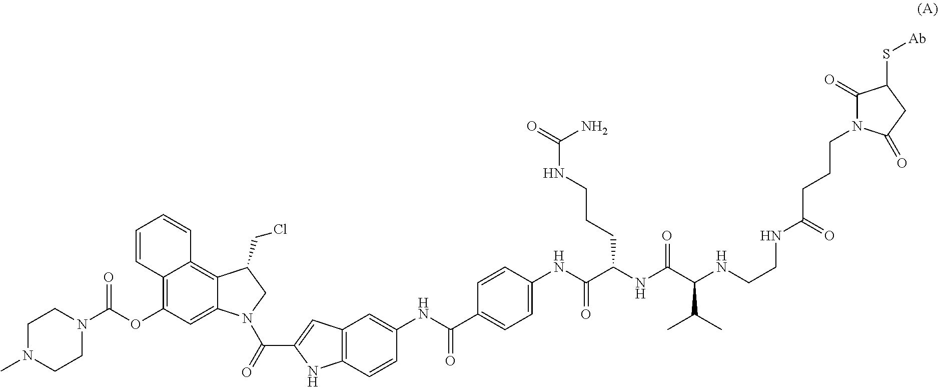

Those skilled in the art will appreciate that structure (m) actually encompasses both the partner molecule (IV) proper and a linker moiety for joining it to the antibody and that, after cleavage of the conjugate, partner molecule (IV) proper is released. Hydrolysis of the carbamate prodrugging group then releases the active molecule proper, as discussed above.

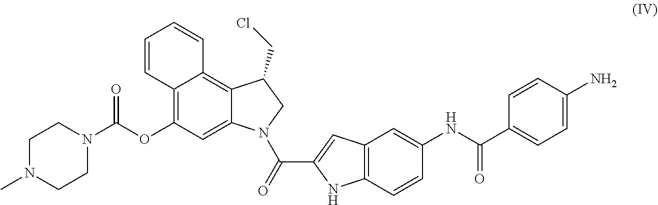

### Example 2

### Tumor-Activated Activity on LNCaP and 786-O Cells

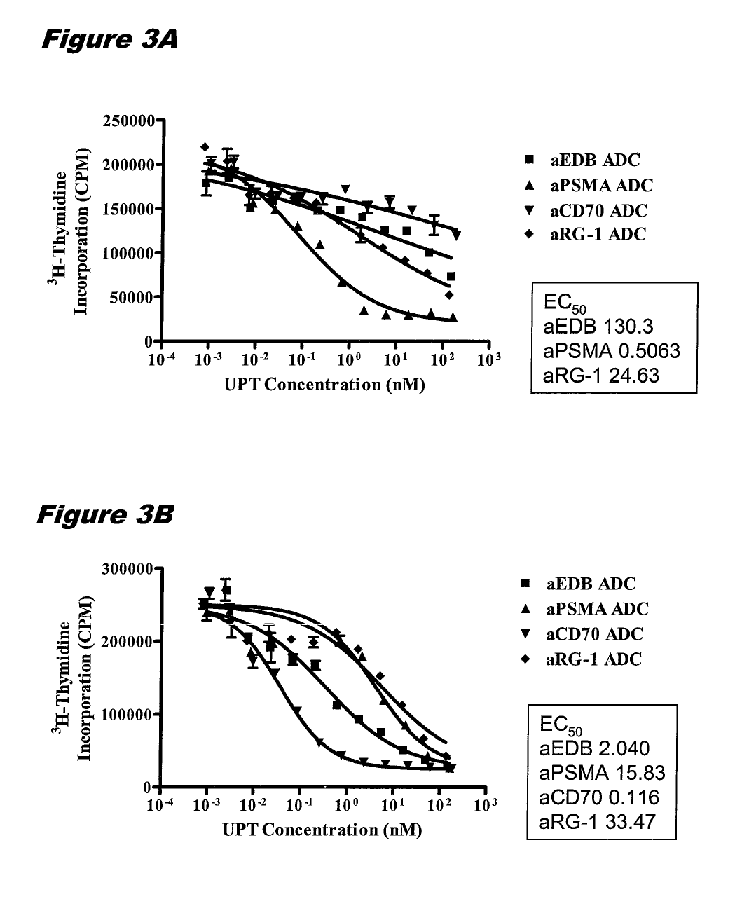

In order to determine the tumor activated activity of anti-RG-1 and ED-B-cytotoxin conjugates, adherent cells, LNCaP (PSMA+/CD70− prostate carcinoma) and 786-O (CD70+/PSMA+ renal cell carcinoma), obtained from ATCC, were cultured in RPMI media containing 10% heat inactivated fetal calf serum (FCS) according to ATCC instructions. The cells were detached from the plate with a trypsin solution. The collected cells were washed and resuspended at a concentration of 0.25 or 0.1×10⁶ cells/ml in RPMI containing 10% FCS for LNCaP and 786-0 cells, respectively. 100 μl of cell suspension were added to 96 well plates and the plates were incubated for 3 hours to allow the cells to adhere. Following this incubation, 1:3 serial dilutions of specific antibody-cytotoxin conjugates starting from 300 nM cytotoxin were added to individual wells. The plates were then incubated for 48 hours, pulsed with 10 μl of a 100 μCi/ml ³H-thymidine and incubated for an additional 24 hours. The plates were harvested using a 96 well Harvester (Packard Instruments) and counted on a Packard Top Count Counter. Four parameter logistic curves were fitted to the ³H-thymidine incorporation as a function of drug molarity using Prism software to determine EC₅₀ values. The logistic curves fitted for the various antibody-cytotoxin conjugates and their resulting EC₅₀ values, in LNCaP and 786-O cells, respectively, are depicted in FIGS. 3A and 3B. Given the difference between the PSMA⁺, RG-1⁺ and ED-B⁺ and CD70⁻ nature of the LNCaP cells and the PSMA⁻, RG-1⁻ and ED-B⁻ and CD70⁺ nature of the 786-0 cells, these graphs indicate that the antibody-cytotoxin conujugates were effective in limiting ³H-thymidine incorporation (and thus indicating decreased growth) in a antigen specific manner.

### Example 3

### Efficacy Against LNCaP/Prostate Stroma Coculture Tumors in SCID Mice

In order to determine the efficacy of anti-RG-1 and ED-B-cytotoxin conjugates of the invention, LNCaP xenografts were performed as follows: 120 CB17.SCID mice were each subcutaneously injected with 2 million LNCaP cells and 1 million prostate stroma cells (cat# CC-2508, Cambrex Bio Science Walkersville, Inc, Walkersville, Md.) resuspended in 0.2 ml of PBS/Matrigel (1:1) (BD Bioscience) at the flank region. This LNCaP/Stroma model expresses high levels of PMSA on the cell surface and high levels of RG-1 in the stroma. CD70 is used as an isotype control as the xenographs are negative for CD70. Mice were weighed and measured for tumors three dimensionally using an electronic caliper once weekly after implantation. Tumor volumes were calculated as height×width×length/2. Mice with tumors averaging 50 mm³ were randomized into 16 treatment groups of seven mice on Day −1 and mice were treated intraperitoneally with vehicle, antibody, or antibody-cytotoxin conjugate according to the dosing regimen described in Table 1 on Day 0, Studies were terminated at Day 62.

TABLE 1
Dosing of SCID Mice

| | Dose (Cytotoxin μmole/kg, Antibody |
|---|---|
| Antibody or Conjugate | mg/kg) |
| Vehicle IP SD (control) | |
| anti-PSMA IP SD | 30 |
| anti-RG-1 IP SD | 30 |
| anti-EDB IP SD | 30 |
| anti-CD70-Toxin IP SD | 0.03, 0.1, 0.3 |
| anti-PSMA-Toxin IP SD | 0.03, 0.1, 0.3 |
| anti-RG-1-Toxin IP SD | 0.03, 0.1, 0.3 |
| anti-EDB-Toxin IP SD | 0.03, 0.1, 0.3 |

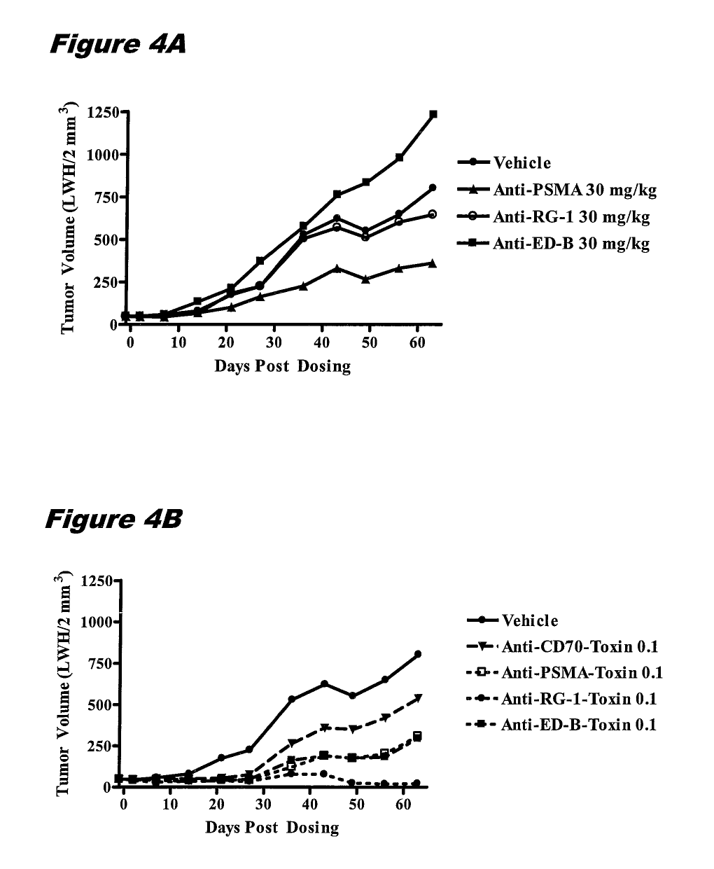

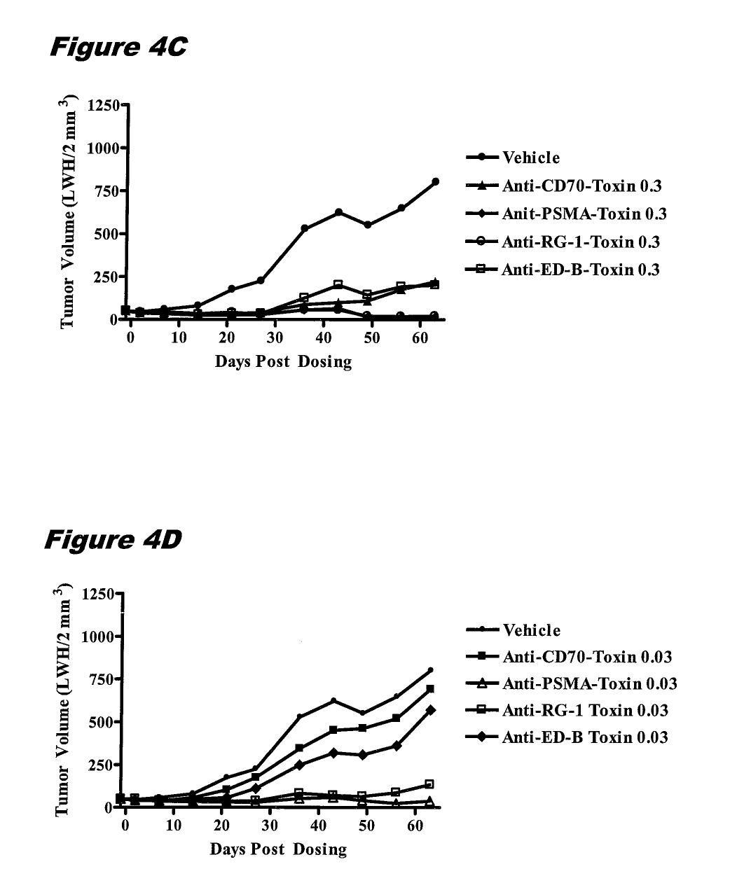

FIGS. 4A through 4D depict the median increase in tumor volume for the seven mice in each of the 16 different groups. As indicated in the top left graph, anti-RG-1 and anti-ED-B naked antibodies had no inhibitory effect on tumor growth. Anti-PSMA naked antibody had some anti-tumor growth effect. This anti-tumor effect was increased upon conjugation of cytotoxin to the anti-PSMA antibody. However, and unexpectedly, anti-tumor activity similar to that of the conjugated antibody to an internalizing antigen was observed when the previously ineffective antibodies to non-internalizing antigens were conjugated to cytotoxin. These results establish that the anti-tumor activity of cytotoxins can be mediated by antibodies to non-internalizing antigens.

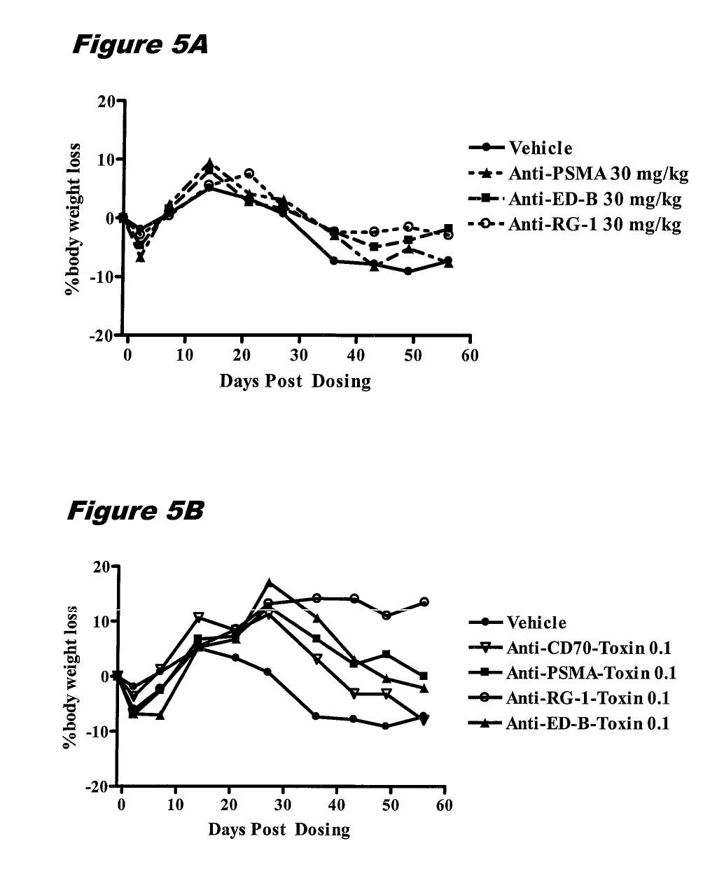

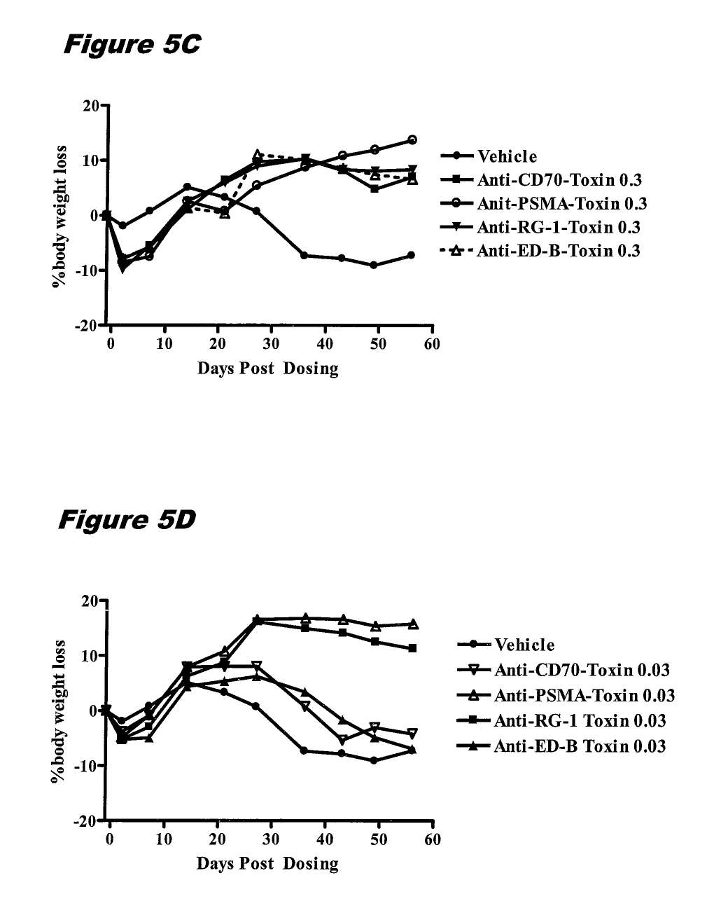

FIGS. 5A through 5D depict the median body weight change for the seven mice in each of the 16 different groups studied. As LNCaP tumors cause cachexia in mice, and this cachexia resulted in weight loss in mice treated with vehicle or naked antibodies, presumably due to tumor growth. In contrast, mice treated with antibody-drug conjugagtes had their lowest body weight right after dosing, indicating that all doses tested 0.03-0.3 were well tolerated. The fact that the mice gained weight in the conjugate groups points to controlled tumor growth and alleviation of cachexia.

### Example 4

### Efficacy Against LNCaP Tumors in SCID Mice

In order to determine the efficacy of anti-RG-1 and ED-B-cytotoxin conjugates of the invention, LNCaP xenografts were performed as follows: 120 CB17.SCID mice were each subcutaneously injected with 2.5 million LNCaP cells resuspended in 0.2 ml of PBS/Matrigel (1:1) (BD Bioscience) at the flank region. Mice were weighed and measured for tumors three dimensionally using an electronic caliper once weekly after implantation. Tumor volumes were calculated as height×width×length/2. This LNCaP model expresses high levels of PMSA on the cell surface and low levels of RG-1 in the stroma. CD70 is used as an isotype control as the xenographs are negative for CD70. Mice with tumors averaging 80 mm³ were randomized into 13 treatment groups of eight mice on Day −1 and mice were treated intraperitoneally with vehicle, antibody, or antibody-toxin conjugate according to the dosing regimen described in Table 2 on Day 0, Studies were terminated at Day 55.

TABLE 2
Dosing of SCID Mice

| | Dose (Cytotoxin μmole/kg, Antibody |
|---|---|
| Antibody or conjugate | mg/kg) |
| Vehicle IP SD (control) | |
| anti-CD70 IP SD | 30 |
| anti-PSMA IP SD | 30 |
| anti-RG-1 IP SD | 30 |
| anti-CD70-Toxin IP SD | 0.03, 0.1, 0.3 |
| anti-PSMA-Toxin IP SD | 0.03, 0.1, 0.3 |
| anti-RG-1-Toxin IP SD | 0.03, 0.1, 0.3 |

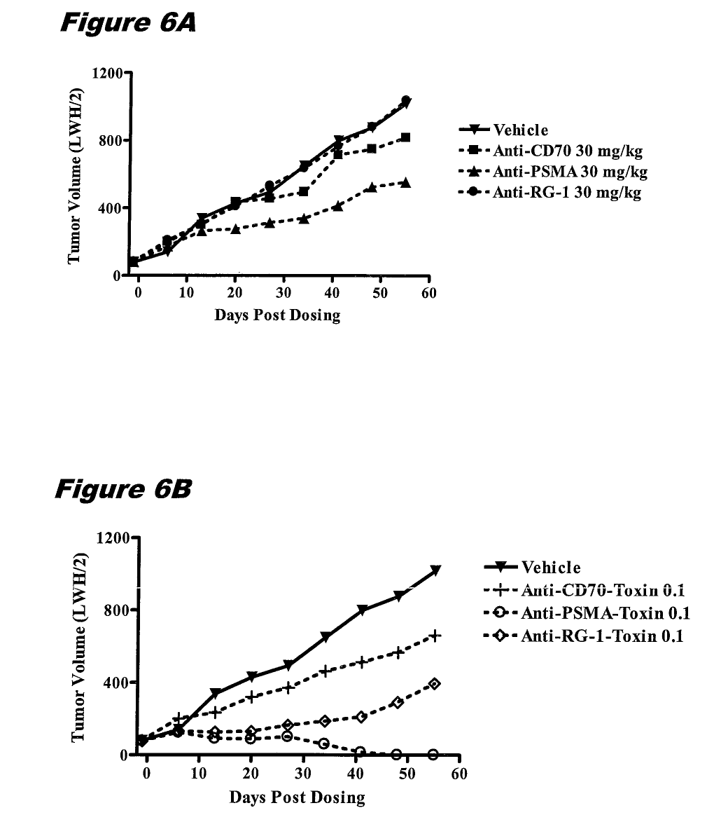

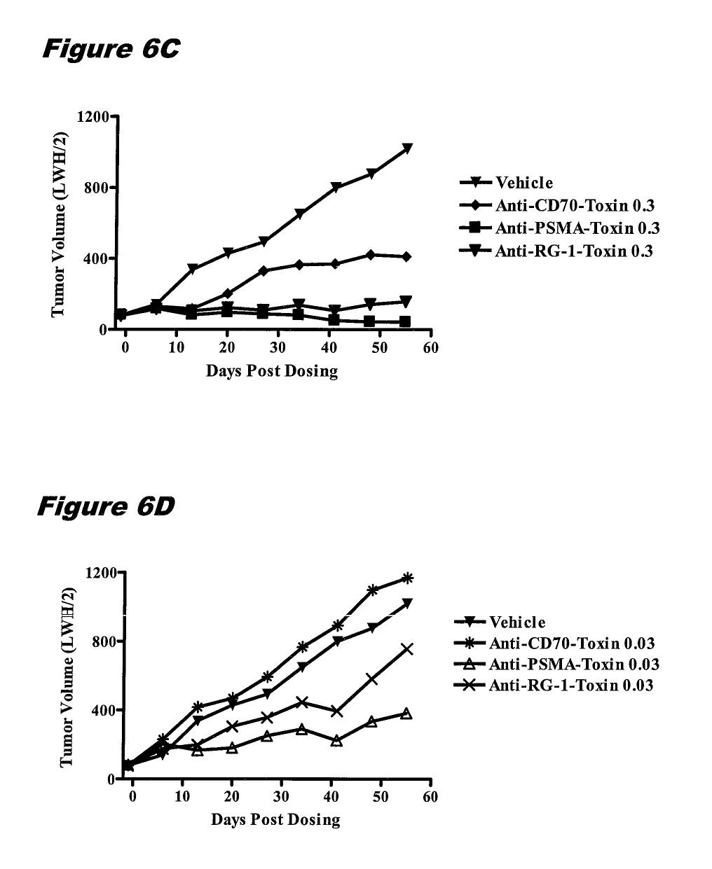

FIGS. 6A through 6D depict the mean increase in tumor volume for the eight mice in each of the 13 different groups studied. Similar to the LNCaP/stroma model, anti-RG-1 naked antibody had no inhibitory effect on tumor growth. Anti-PSMA naked antibody had some anti-tumor growth effect. The anti-PSMA antibody's anti-tumor effect was markedly increased upon conjugation of cytotoxin. A similar, but unexpected, anti-tumor activity was observed when non-internalizing anti-RG-1 antibody was conjugated to cytotoxin. These results further confirm that the anti-tumor activity of cytotoxins can be mediated by antibodies to non-internalizing antigens.

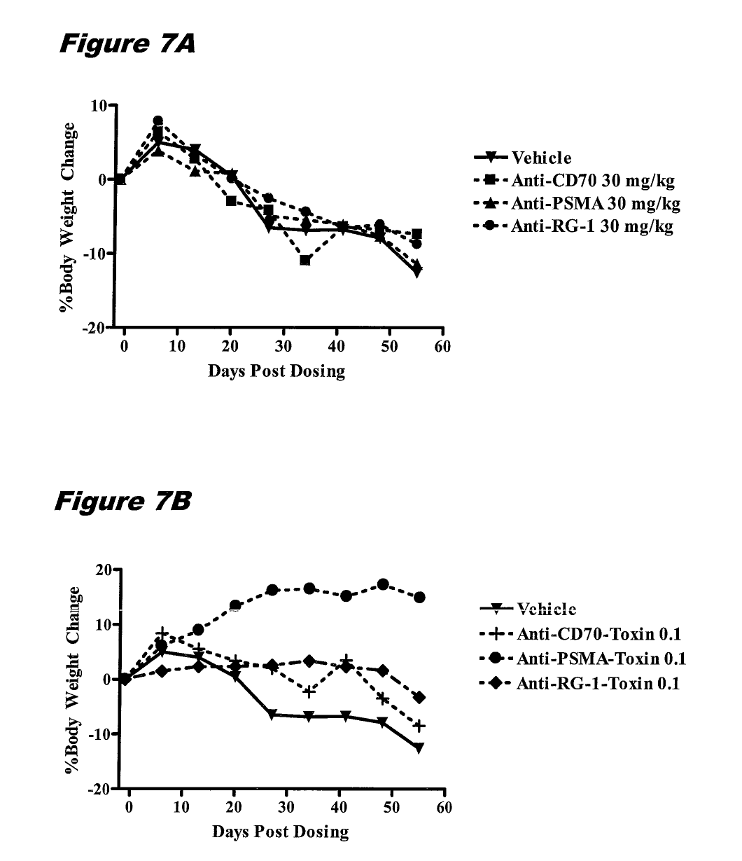

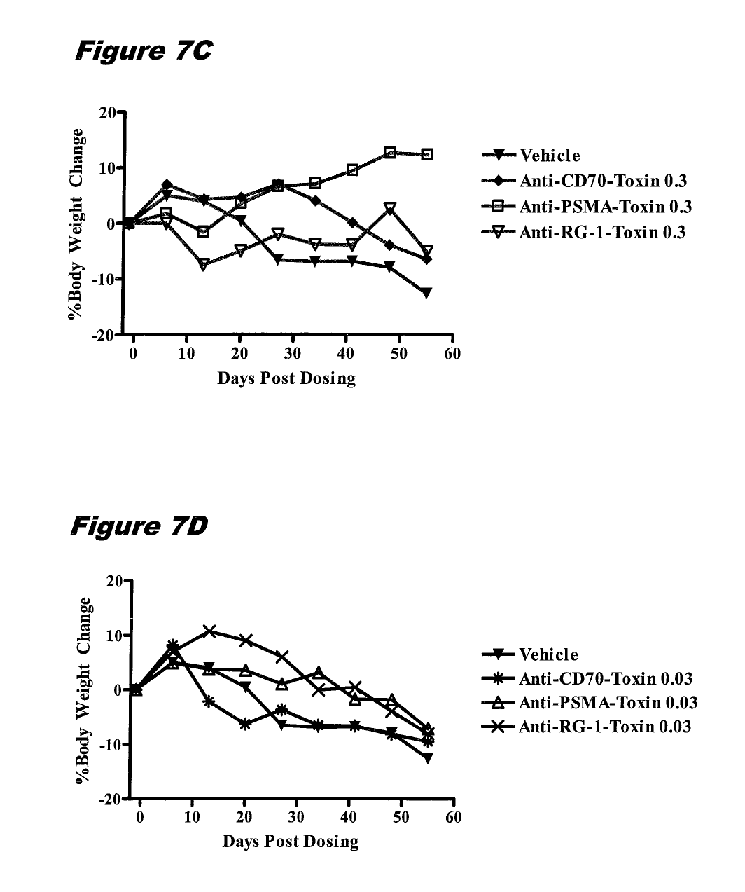

FIGS. 7A through 7D depict the median body weight change for the eight mice in each of the 16 different groups. As pointed out above, LNCaP tumors cause cachexia in mice. Again, this cachexia resulted in weight loss in mice treated with vehicle or naked antibodies, presumably due to increased tumor growth. In contrast, mice treated with antibody-drug conjugates had their lowest body weight right after dosing, indicating that all doses we tested 0.03-0.3 were well tolerated. The fact that the mice gained weight in the conjugate groups points to control of tumor growth and alleviation of cachexia.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

SUMMARY OF SEQUENCE LISTINGS

| SEQ ID NO: | SEQUENCE |
|---|---|
| 1 | V_{H} CDR1 a.a. 19G9 |
| 2 | V_{H} CDR1 a.a. 34E1 |
| 3 | V_{H} CDR2 a.a. 19G9 |
| 4 | V_{H} CDR2 a.a. 34E1 |
| 5 | V_{H} CDR3 a.a. 19G9 |
| 6 | V_{H} CDR3 a.a. 34E1 |
| 7 | V_{L} CDR1 a.a. 19G9 |
| 8 | V_{L} CDR1 a.a. 34E1 |
| 9 | V_{L} CDR2 a.a. 19G9 |
| 10 | V_{L} CDR2 a.a. 34E1 |
| 11 | V_{L} CDR3 a.a. 19G9 |
| 12 | V_{L} CDR3 a.a. 34E1 |
| 13 | V_{H} a.a. 19G9 |
| 14 | V_{H} a.a. 34E1 |
| 15 | V_{L} a.a. 19G9 |
| 16 | V_{L} a.a. 34E1 |
| 17 | V_{H} n.t. 19G9 |
| 18 | V_{H} n.t. 34E1 |
| 19 | V_{L} n.t. 19G9 |
| 20 | V_{L} n.t. 34E1 |
| 21 | Peptide Linker |
| 22 | Peptide Linker |
| 23 | Peptide Linker |
| 24 | Peptide Linker |
| 25 | Peptide Linker |
| 26 | Peptide Linker |
| 27 | Peptide Linker |
| 28 | Peptide Linker |
| 29 | Peptide Linker |
| 30 | Peptide Linker |

1. An antibody-partner molecule conjugate comprising a human monoclonal antibody or an antigen-binding portion thereof conjugated to a partner molecule, wherein the antibody or antigen-binding portion thereof binds human RG-1 and the conjugate exhibits at least one of the following properties:
(a) binds to human RG-1 with a K_{D} of 1×10⁻⁸ M or less; or
(b) inhibits growth of RG-1-expressing cells in vivo.

2. The conjugate of claim 1, wherein the antibody or antigen-binding portion thereof comprises:
(a) a heavy chain variable region CDR1 comprising SEQ ID NO: 1;
(b) a heavy chain variable region CDR2 comprising SEQ ID NO: 3;
(c) a heavy chain variable region CDR3 comprising SEQ ID NO: 5;
(d) a light chain variable region CDR1 comprising SEQ ID NO: 7;
(e) a light chain variable region CDR2 comprising SEQ ID NO: 9; and
(f) a light chain variable region CDR3 comprising SEQ ID NO: 11.

3. The conjugate of claim 1, wherein the antibody or antigen-binding portion thereof comprises:
(a) a heavy chain variable region CDR1 comprising SEQ ID NO: 2;
(b) a heavy chain variable region CDR2 comprising SEQ ID NO: 4;
(c) a heavy chain variable region CDR3 comprising SEQ ID NO: 6;
(d) a light chain variable region CDR1 comprising SEQ ID NO: 8;
(e) a light chain variable region CDR2 comprising SEQ ID NO: 10; and
(f) a light chain variable region CDR3 comprising SEQ ID NO: 12.

4. The conjugate of claim 1, wherein the antibody or antigen-binding portion thereof comprises:
(a) a heavy chain variable region comprising an amino acid sequence selected from the group consisting of SEQ ID NOs:13-14 or conservative modifications of either; and
(b) a light chain variable region comprising an amino acid sequence selected from the group consisting of SEQ ID NOs:15-16 or conservative modifications of either.

5. The conjugate of claim 1, wherein the antibody or antigen-binding portion thereof comprises:
(a) a heavy chain variable region CDR1 comprising SEQ ID NO: 1 or SEQ ID NO: 2 or conservative modifications of either;
(b) a heavy chain variable region CDR2 comprising SEQ ID NO: 3 or SEQ ID NO: 4 or conservative modifications of either;
(c) a heavy chain variable region CDR3 comprising SEQ ID NO: 5 or SEQ ID NO: 6 or conservative modifications of either;
(d) a light chain variable region CDR1 comprising SEQ ID NO: 7 or SEQ ID NO: 8 or conservative modifications of either;
(e) a light chain variable region CDR2 comprising SEQ ID NO: 9 or SEQ ID NO: 10 or conservative modifications of either; and
(f) a light chain variable region CDR3 comprising SEQ ID NO: 11 or SEQ ID NO: 12 or conservative modifications of either.

6. The conjugate of claim 1, wherein the antibody or antigen-binding portion thereof binds to an epitope on human RG-1 recognized by a reference antibody comprising:
(a) a heavy chain variable region comprising the amino acid sequence of SEQ ID NO:13 and a light chain variable region comprising the amino acid sequence of SEQ ID NO:15; or
(b) a heavy chain variable region comprising the amino acid sequence of SEQ ID NO:14 and a light chain variable region comprising the amino acid sequence of SEQ ID NO:16.

7. (canceled)

8. The conjugate of claim 1, wherein the partner molecule has a structure represented by formula (I): 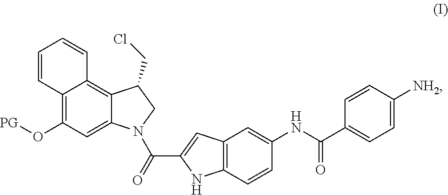
wherein PD represents a prodrugging group selected from the group consisting of a carbamate, a phosphate, or a β-glucuronic acid derivative.

9-11. (canceled)

12. The conjugate of claim 1, having a structure represented by formula (A): 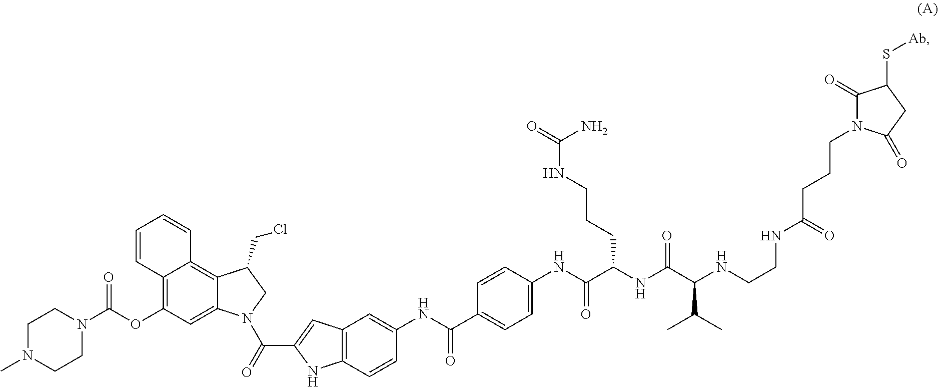
wherein Ab represents the antibody or antigen binding portion thereof.

13. A composition comprising the conjugate of claim 1 and a pharmaceutically acceptable carrier.

14. A method of inhibiting the growth of a RG-1-expressing tumor cell, comprising contacting the RG-1-expressing tumor cell with a conjugate according claim 1 such that growth of the RG-1-expressing tumor cell is inhibited.

15-17. (canceled)

18. The method of claim 14, wherein the conjugate has a structure represented by formula (A): 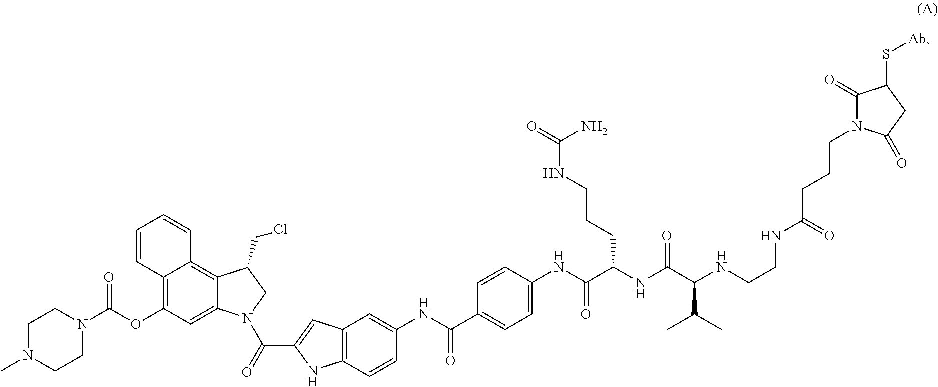
wherein Ab represents the antibody or antigen binding portion thereof.

19. The method of claim 14, wherein the RG-1-expressing tumor cell is a prostate cancer or bladder cancer cell.

20. A method of treating cancer in a subject comprising administering to a subject in need of such treatment an effective amount of a conjugate according to claim 1 such that the cancer is treated in the subject.

21. The method of claim 20, wherein the cancer is prostate cancer or bladder cancer.

22. The method of claim 20, wherein the cancer is lung cancer, gastric cancer, breast cancer, renal cancer, pancreatic cancer, colon cancer, melanoma, or insulinoma.

23-27. (canceled)


SEQUENCE LISTING (30 sequences)

SEQ ID NO: 1 - protein, 5 aa, Homo sapiens
SYVMH

SEQ ID NO: 2 - protein, 5 aa, Homo sapiens
SYVMH

SEQ ID NO: 3 - protein, 16 aa, Homo sapiens
VIGTGGVTHYADSVKG

SEQ ID NO: 4 - protein, 16 aa, Homo sapiens
VIGTGGVTNYADSVKG

SEQ ID NO: 5 - protein, 16 aa, Homo sapiens
WGYYGSGSYENDAFDI

SEQ ID NO: 6 - protein, 10 aa, Homo sapiens
WGDWDDAFDI

SEQ ID NO: 7 - protein, 12 aa, Homo sapiens
RASQSVSSSYLA

SEQ ID NO: 8 - protein, 12 aa, Homo sapiens
RASQSVSSSYLA

SEQ ID NO: 9 - protein, 7 aa, Homo sapiens
GASSRAT

SEQ ID NO: 10 - protein, 7 aa, Homo sapiens
GASSRAT

SEQ ID NO: 11 - protein, 8 aa, Homo sapiens
QQYSSSLT

SEQ ID NO: 12 - protein, 8 aa, Homo sapiens
QQYGSSLT

SEQ ID NO: 13 - protein, 124 aa, Homo sapiens
EVQLVQSGGGLVHPGGSLRLSCAGSGFTFSSYVMHWLRQAPGKGLEWVSVIGTGGVTHYADSVKGRFTISRDNAKNSLYLQMNSLRAEDMAMYYCARWGYYGSGSYENDAFDIWGQGTMVTVSS

SEQ ID NO: 14 - protein, 118 aa, Homo sapiens
EVQLVQSGGGLVHPGGSLRLSCAGSGFTFSSYVMHWVRQAPGKGLEWVSVIGTGGVTNYADSVKGRFTISRDNAKNSLYLQMNSLRAEDMAVYYCARWGDWDDAFDIWGQGTMVTVSS

SEQ ID NO: 15 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSLTFGGGTKVEIK

SEQ ID NO: 16 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIK

SEQ ID NO: 17 - DNA, 372 bp, Homo sapiens
gaggttcagctggtgcagtctgggggaggcttggtacatcctggggggtccctgagactctcctgtgcaggctctggattcaccttcagtagctatgttatgcactggcttcgccaggctccaggaaaaggtctggagtgggtatcagttattggtactggtggtgtcacacactatgcagactccgtgaagggccgattcaccatctccagagacaatgccaagaactccttgtatcttcaaatgaacagcctgagagccgaggacatggctatgtattactgtgcaagatggggttactatggttcggggagttatgagaatgatgcttttgatatctggggccaagggacaatggtcaccgtctcttca

SEQ ID NO: 18 - DNA, 354 bp, Homo sapiens
gaggttcagctggtgcagtctgggggaggcttggtacatcctggggggtccctgagactctcctgtgcaggctctggattcaccttcagtagctatgtcatgcactgggttcgccaggctccaggaaaaggtctggagtgggtatcagtaattggtactggtggtgtcacaaactatgcagactccgtgaagggccgattcaccatctccagagacaatgccaagaactccttgtatcttcaaatgaacagcctgagagccgaggacatggctgtgtattactgtgcaagatggggggactgggatgatgcttttgatatctggggccaagggacaatggtcaccgtctcttca

SEQ ID NO: 19 - DNA, 321 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatagtagctcgctcactttcggcggggggaccaaggtggagatcaaa

SEQ ID NO: 20 - DNA, 321 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaggtggagatcaaa

SEQ ID NO: 21 - protein, 6 aa, Artificial Sequence
  Peptide linker
PVGLIG

SEQ ID NO: 22 - protein, 5 aa, Artificial Sequence
  Peptide linker
GPLGV

SEQ ID NO: 23 - protein, 8 aa, Artificial Sequence
  Peptide linker
GPLGIAGQ

SEQ ID NO: 24 - protein, 4 aa, Artificial Sequence
  Peptide linker
PLGL

SEQ ID NO: 25 - protein, 8 aa, Artificial Sequence
  Peptide linker
GPLGMLSQ

SEQ ID NO: 26 - protein, 8 aa, Artificial Sequence
  Peptide linker
GPLGLWAQ

SEQ ID NO: 27 - protein, 4 aa, Artificial Sequence
  Peptide linker
XLAL

SEQ ID NO: 28 - protein, 4 aa, Artificial Sequence
  Peptide linker
GFLG

SEQ ID NO: 29 - protein, 4 aa, Artificial Sequence
  Peptide linker
LLGL

SEQ ID NO: 30 - protein, 4 aa, Artificial Sequence
  Peptide linker
XLAL